(19) United States
(12) Patent Application Publication
(10) Pub. No.: US 2022/0175285 A1
(43) Pub. Date: Jun. 9, 2022

(54) TRANSLATIONAL METHOD TO DETERMINE BIOLOGICAL BASIS FOR DYSLEXIA

(71) Applicant: Lafayette College, Easton, PA (US)

(72) Inventor: Lisa Ann Gabel, Schnecksville, PA (US)

(21) Appl. No.: 17/652,003

(22) Filed: Feb. 22, 2022

Related U.S. Application Data
(63) Continuation of application No. 15/934,567, filed on Mar. 23, 2018.
(60) Provisional application No. 62/475,553, filed on Mar. 23, 2017.

(51) Int. Cl. A61B 5/16; G09B 5/08; G16H 50/20; G16H 50/30

(52) U.S. Cl. CPC A61B 5/16; G09B 5/08; Y02A 90/10; G16H 50/30; G16H 50/20

(57) ABSTRACT

A virtual Hebb-Williams maze task, comprising at least four of Hebb-Williams mazes selected from the group consisting of mazes 5, 6, 8, 11, and 12; a computer implemented software capable of detecting pointer position according to a coordinate system for tracking the position and time of the user; and wherein the position and time of the user can be annotated to define a score which can be compared to a control; and wherein a score of more than one standard deviation below the control identifies a child at risk for Reading Disorder.

### CROSS-REFERENCE TO RELATED APPLICATIONS

This application is a continuation of U.S. patent application Ser. No. 15/934,567 filed on Mar. 23, 2018, which claims the benefit of U.S. Provisional Patent Application No. 62/475,553 filed on Mar. 23, 2017, with the United States Patent and Trademark Office, the contents of which are incorporated herein by reference in their entirety.

### GOVERNMENT FUNDING

This invention was made with government support under Grant No. HD087937 awarded by the National Institutes of Health. The government has certain rights in the invention.

### FIELD OF THE INVENTION

This invention is directed toward methods and systems for using virtual Hebb-Williams mazes to detect levels of reading impairment in children.

### BACKGROUND OF THE INVENTION

Dyslexia, or Reading Disability (RD), is a specific impairment in processing written language despite adequate intelligence and educational background. RD, affecting 5-17% of school age children, has far-reaching social and economic consequences. Early detection and intervention will help to close the gap between typically developing and reading impaired children in acquiring reading skills. One of the challenges with early detection is that reading is a complex process, and difficulties with reading may arise from deficits in one or more cognitive processes. A stronger understanding of an individual student's specific challenges may allow for interventions tailored to meet a student's unique learning needs. For nearly three decades, the phonological model of dyslexia was considered the dominant model, however the phonological basis of dyslexia is predominantly based on data from English, a nontransparent language orthography. However, children from more transparent language orthographies (i.e., one-to-one grapheme-phoneme correspondence), such as German, exhibit fewer phonemic errors when learning to read compared to children from nontransparent languages (Landerl, et al. 1997). In addition, recent studies have provided evidence that visuo-spatial attention may be a core deficit of dyslexia. This is consistent with neurophysiological evidence and neuroanatomical data demonstrating developmental disruptions in visual systems. Currently however, there are a lack of instruments that efficiently measure visual spatial processes which underlie reading. In addition, current reading measures rely heavily on oral reporting and the use of letters to assess reading ability making it challenging to determine whether language learners, such as English language learners have an intrinsic learning disability or lack exposure to written and oral English language. The current measure is a nonlanguage based assessment which focuses on core deficit of dyslexia that is currently not being examined and can be used to assess both native speakers and language learners.

If learning disabilities remain untreated, a child may experience long term social and emotional problems which influence future success in all aspects of their lives. Therefore, detection and treatment of RD are vital to reducing the effects of RD on a child. This work directly aligns with the mission of NICHD to ensure “that all children have the chance to achieve their full potential for healthy and productive lives.” The complete explanation of a complex neurodevelopmental disorder requires an understanding across multiple levels, including, but not limited to, cognition, behavior, and genetics. Although our understanding and treatment for dyslexia has greatly increased in the last 20 years, a significant percentage of children with dyslexia are either identified too late or have a specific manifestation of the disorder that is not understood well enough to design and deliver a successful remediation (1). Research examining the connection among genetic, cognitive and behavioral aspects of reading disorder offers promise for early identification and intervention to successfully address specific phenotypes of RD (2).

Dyslexia involves poor literacy skills (i.e., reading accuracy and/or fluency) despite adequate intelligence and educational opportunities (3-5). Dyslexia presents with similar cognitive, neuroanatomical and genetic traits despite additional spelling and writing impairments associated with reading disorder, therefore these two terms will be considered synonymous. RD is a phenotypically complex developmental disorder with a significant genetic component. As the most common learning disability (6), RD is a global issue that affects 5-17% of the world's population. Therefore, RD has far-reaching social and economic consequences. Several cognitive and perceptual changes appear to associate with RD including changes in short-term memory, oculomotor skills, visuospatial abilities, sensory processing, semantic encoding, integration of letter and speech sounds, and phonological processing (7-22). It remains controversial as to whether all of these features are central to the core RD phenotype; however, the majority of available data point to phonological and visual deficits.

With an approximate 60% heritability rate, genetic linkage and association studies were carried out in dyslexic families across Finland, Germany, United Kingdom, and the United States resulting in the identification of several candidate dyslexia susceptibility genes (CDSGs). The most replicated CDSGs are DCDC2 and KIAA0319 within the DYX2 locus (for review see [19-21, 23]). Recently, specific risk variants for RD within the DYX2 locus (24) have been identified and associated with RD severity (25, 26). Altered neural connectivity (27) and functional neural activity (28) within temporoparietal regions of the brain are hypothesized to be associated with altered expression of DCDC2 and KIAA0319 in individuals with RD (29).

Genetic knockdown (30-36) and knockout studies (30, 37, 38) in animals have provided evidence that disruption in CDSG expression of Dcdc2, Dyx1c1, and Kiaa0319 results in varying degrees of altered neuronal migration, as well as cortical and sensory processing deficits (31, 32, 36, 37). Kiaa0319 (39) and Dcdc2 (38) have recently been shown to disrupt visuospatial learning and memory in knockout mice.

Utilization of animal models is an efficient and practical way to address complex questions regarding the link between genes and behavior. However, gene-environment interactions and developmental processes that are involved in human disorders may not be perfectly modeled in different species. In order to make accurate cross-species comparisons, we must correctly model the human system. Virtual environments, which mimic physical mazes used with animal models, enable us to translate research conducted in the laboratory to human participants.

Certain maze tasks have been successfully created on a virtual platform to make direct comparisons between animal models and humans (40-46). Accordingly a new model is necessary to utilize certain systems and methods of virtual mazes to identify RD at an early age, whereby early detection provides for an avenue for treatment of the individual and can assist with reducing the effects of RD as the child progresses through educational years.

### SUMMARY OF THE INVENTION

The embodiments herein are directed toward a virtual Hebb-Williams (vHW) maze task for use as a low-cost, time efficient, and easy-to-use assessment for the early detection of children at risk for reading impairment. The vHW maze offers the potential to serve as a reliable, nonlanguage based predictor of reading difficulty which can improve early identification and intervention efforts for young children. Unlike current screening measures of reading, the vHW maze could be administered in the classroom, with a fully integrated analytical system. This tool will provide valuable information about a child's learning processes that could inform instructional practice. With the successful attainment of this research program, the vHW maze task will fill important gaps in early identification screeners, by examining a broader range of cognitive processes associated with reading and enhancing our understanding factors underlying reading impairment.

Therefore, In accordance with these and other objects, a further embodiment is directed to a system comprising a virtual Hebb-Williams maze task, comprising at least four of Hebb-Williams mazes selected from the group consisting of Mazes 5, 6, 8, 11, and 12; a computer implemented software capable of detecting pointer position according to a coordinate system for tracking the position and time of the user; and wherein the position and time of the user can be annotated to define a score which can be compared to a control; and wherein a score of more than one standard deviation below the control identifies a child at risk for Reading Disorder.

A system comprising a virtual Hebb-Williams maze task, comprising at least four of Hebb-Williams mazes selected from the group consisting of Mazes 5, 6, 8, 11, and 12; a computer implemented software capable of detecting a pointer position according to a coordinate system along the x- and y-axes for tracking the position and time of the user; and wherein the position and time of the user can be annotated to define a score which can be compared to a control; and wherein a score of more than one standard deviation below the control identifies a child at risk for Reading Disorder. In preferred embodiments, the system wherein each maze is a 6×6 grid having a total of 36 cells, said cells arranged according to a Cartesian plot of A-F in the x-axis, 1-6 in the y-axis, and vertical walls in the z-axis. In preferred embodiments, the system wherein each cell comprises 256 units, each unit having a length of 1 cm. In preferred embodiments, the system wherein a virtual path through the 6×6 grid takes no more than 120 seconds.

In certain preferred embodiments, a system of the above wherein each of the Mazes 5, 6, 8, 11, and 12 have the following internal walls and most efficient solutions: Maze 5, external walls, and internal walls from: A1/A2, A2/B2, A3/B3, B3/B4, C3/C4, B1/C1, C1/C2, D1/D2, C2/D2, and C3/D3, C4/D4, C5/D5, with the most direct path from F6, E6, D5, D4, C3, B2, A1; Maze 6, and internal walls from A1/A2, B1/B2, C1/C2, D1/D2, E1/D2, F2/F3, and A3/A4, B3/B4, C3/C4, D3/D4, D4/E4, D5/E5 with the most direct path is F6, F5, F4, F3, E2, F1, E1, D1, C1, B1, and A1; Maze 8 having internal walls from A1/B1, A2/B2, A3/B3, B3/B4, A5/A4, B5/B4, C5/C4, D5/D4, E5/E4, E4/F4, and E3/E4; with the shortest solution is F6, F5, F4, E3, D4, C4, B4, A3, A2, and A1; Maze 11 internal walls at A1/A2, B1/B2, C1/C2, C2/D2, C3/D3, F1/F2, E1/E2, D2/E2, D3/E3, D4/F4, A4/B4, A5/B5, and B3/C3, B4, C4; with the shortest solution is F6, E5, D4, D3, D2, C1, B1, and A1; and Maze 12 with internal walls at A1/B1, A2/B2, A3/B3, A4B4, A5/B5, B5/B6, B2/C2, B3/C3, B4/C4, and D5/E5, D6/E6; and the shortest solution is from F6, E5, D4, C5, B6, A5, A4, A3, A2, and A1.

A method of detecting reading disability (RD) in a child comprising administering to said child a set of at least 4 Hebb-Williams virtual mazes; wherein each maze is completed a total of six times, each completion taking no more than 120 seconds; and after completion of each maze, a gap of about 120 seconds between a maze and a subsequent maze; annotating the position of a cursor according to a coordinate system and time, calculating a score for the child for each completed maze, and wherein a calculated score can be compared to a predetermined control score to predict the presence of a reading disorder in said child. In a preferred embodiment, the method further comprising wherein a detection of a score more than one standard deviation below a control indicates a reading disorder wherein the child is treated for a reading disorder. In a preferred embodiment, the method further comprising wherein each Hebb-Williams virtual maze is a 6×6 grid having a total of 36 cells, said cells arranged according to a Cartesian plot of A-F in the x-axis, 1-6 in the y-axis, and vertical walls in the z-axis. In a further embodiment, the method further comprising wherein each cell comprises 256 units, each unit having a length of 1 cm. In a preferred embodiment, the method wherein a virtual path through the 6×6 grid takes no more than 120 seconds.

In a preferred embodiment, the method as above wherein a first Hebb-Williams virtual maze is Hebb-Williams Maze 1, wherein a child is oriented to starting from a start and finishing at a finish in Maze 1. In a preferred embodiment, the method wherein subsequent to orienting the child on Maze 1, a child is tested on at least four of Hebb-Williams Mazes 5, 6, 8, 11, and 12. In a preferred embodiment, the method wherein Mazes 5, 6, 8, 11, and 12 have the following internal walls and most efficient solutions: Maze 5, external walls, and internal walls from: A1/A2, A2/B2, A3/B3, B3/B4, C3/C4, B1/C1, C1/C2, D1/D2, C2/D2, and C3/D3, C4/D4, C5/D5, with the most direct path from F6, E6, D5, D4, C3, B2, A1; Maze 6, and internal walls from A1/A2, B1/B2, C1/C2, D1/D2, E1/D2, F2/F3, and A3/A4, B3/B4, C3/C4, D3/D4, D4/E4, D5/E5 with the most direct path is F6, F5, F4, F3, E2, F1, E1, D1, C1, B1, and A1; Maze 8 having internal walls from A1/B1, A2/B2, A3/B3, B3/B4, A5/A4, B5/B4, C5/C4, D5/D4, E5/E4, E4/F4, and E3/E4; with the shortest solution is F6, F5, F4, E3, D4, C4, B4, A3, A2, and A1; Maze 11 internal walls at A1/A2, B1/B2, C1/C2, C2/D2, C3/D3, F1/F2, E1/E2, D2/E2, D3/E3, D4/F4, A4/B4, A5/B5, and B3/C3, B4, C4; with the shortest solution is F6, E5, D4, D3, D2, C1, B1, and A1; and Maze 12 with internal walls at A1/B1, A2/B2, A3/B3, A4B4, A5/B5, B5/B6, B2/C2, B3/C3, B4/C4, and D5/E5, D6/E6; and the shortest solution is from F6, E5, D4, C5, B6, A5, A4, A3, A2, and A1.

In a preferred embodiment, a virtual reading disability quantification system comprising a set of six mazes, a first maze indicated for training, each maze having exterior walls and internal walls as delineated within the exterior of a 6×6 grid with a cartesian plot of A-F along the x-axis and 1-6 along the y-axis and walls extending vertically in the z-axis, with Maze 1 having internal walls along the border of the following cells: C1/D1, C2/D2, E1/F1, E2/F2, E3/F3, E4/F4, A3/A4, B3/B4, A5/A6, B5/B6, C5/C6, and D5/D6; with a start at F6, and a finish at A1, having the most efficient path to traverse F6, E5, D4, C3, B2, and A1 on the diagonal; and four additional mazes selected from the group consisting of: Maze 5, external walls, and internal walls from: A1/A2, A2/B2, A3/B3, B3/B4, C3/C4, B1/C1, C1/C2, D1/D2, C2/D2, C3/D3, C4/D4, C5/D5, with the most direct path from F6, E6, D5, D4, C3, B2, A1; Maze 6, and internal walls from A1/A2, B1/B2, C1/C2, D1/D2, E1/D2, F2/F3, and A3/A4, B3/B4, C3/C4, D3/D4, D4/E4, D5/E5 with the most direct path is F6, F5, F4, F3, E2, F1, E1, D1, C1, B1, and A1; Maze 8 having internal walls from A1/B1, A2/B2, A3/B3, B3/B4, A5/A4, B5/B4, C5/C4, D5/D4, E5/E4, E4/F4, and E3/E4; with the shortest solution is F6, F5, F4, E3, D4, C4, B4, A3, A2, and A1; Maze 11 internal walls at A1/A2, B1/B2, C1/C2, C2/D2, C3/D3, F1/F2, E1/E2, D2/E2, D3/E3, D4/F4, A4/B4, A5/B5, and B3/C3, B4, C4; with the shortest solution is F6, E5, D4, D3, D2, C1, B1, and A1; and Maze 12 with internal walls at A1/B1, A2/B2, A3/B3, A4B4, A5/B5, B5/B6, B2/C2, B3/C3, B4/C4, and D5/E5, D6/E6; and the shortest solution is from F6, E5, D4, C5, B6, A5, A4, A3, A2, and A1.

In a preferred embodiment, the system wherein each Maze is completed a total of six times, each completion taking no more than 120 seconds; and after completion of each maze, a gap of about 120 seconds between a maze and a subsequent maze. In a preferred embodiment, the system wherein the system annotates the position of a cursor according to the coordinate system and time and calculates a score for the child for each completed maze, and wherein a calculated score can be compared to a predetermined control score to predict the presence of a reading disorder in said child. In a preferred embodiment, the system wherein each maze is completed a total of six times, each completion taking no more than 120 seconds; and after completion of each maze, a gap of about 120 seconds between a maze and a subsequent maze; annotating the position of a cursor according to a coordinate system and time, calculating a score for the child for each completed maze, and wherein a calculated score can be compared to a predetermined control score to predict the presence of a reading disorder in said child. In a preferred embodiment, the system wherein a score of more than one standard deviation below the control identifies a child at risk for Reading Disorder.

A method of detecting RD in a child comprising administering to said child a set of at least 4 Hebb-Williams virtual mazes; wherein each maze is completed a total of six times, each completion taking no more than 120 seconds; and after completion of each maze, a gap of about 120 seconds between a maze and a subsequent maze; annotating the position of a cursor according to a coordinate system and time, calculating a score for the child for each completed maze, and wherein a calculated score can be compared to a predetermined control score to predict the presence of a reading disorder in said child. In certain embodiments, a detection of a statistical average performance efficiency score, across 6 trials on at least two maze tasks, of greater than 0 would indicate that the child is significantly impaired at this task in comparison to a child who has developed typical reading strategies. More specifically, a positive performance efficiency score would indicate that the child made more errors and took longer to complete the task for any given trial; although learning occurs, as expected, it will be at a slower rate. This impaired maze learning performance is indicative of a reading impairment wherein the child is treated for a reading disorder.

An electronic game capable of determining or assisting with evaluation of a learning disorder comprising a system as described above.

### BRIEF DESCRIPTION OF THE FIGURES

FIGS. 1A-1C depict various aspects of virtual Hebb-Williams mazes. FIG. 1A is a schematic of maze configuration 1. The maze is partitioned into 36 cells (A1 to F6), creating a 6×6 configuration in the x- and y-planes. Each cell consists of 256 units within the Unreal software. Accordingly, each cell can be further broken down into these additional 256 units (a 16×16 set of boxes) in each of the cells. A unit within the Unreal software is approximately equal to 1 cm. The individual must navigate through the maze from the start box (S) to the goal box (G) in the x- and y-planes. The z-axis of the 3D maze consist of the walls which define the boundary of the maze and the interior corridors and dead-end zones. The heavy solid black lines in the schematic represent the walls within the maze. The grid lines are shown as the cells within the maze. The bold lines depict the error zones, which are not visible to the participant, but mark the paths within the maze which will not lead to the goal box. The goal box (G) is the site of the ball (the target) which the participant must locate. The goal and start boxes are each 300 units deep. The participant has a viewing height (eye level) of 85 units from the ground. The walls extend above the viewing height along the z-axis, which creates the 3D experience for the individual navigating through the maze. FIG. 1B is a schematic of HW Mazes 1, 5, 6, 8, 11, and 12. Individual dots indicate location of first person when traveling through Maze 5 as pictured in FIG. 1B. The lines represent walls, the dotted lines represent error zones (paths which do not lead to the target). FIG. 1C depicts scenes from virtual HW Maze 5: entering the maze, reaching a dead end, and identifying the goal box. If the participant did not locate the target in 120 s, arrows appeared to guide the participant from the start box to the goal box. Data were no longer collected after 120 s had elapsed from the time the participant entered the maze.

FIGS. 2A and 2B show representative paths taken by children completing the Hebb-Williams maze configurations. Composite traces from all six trials for Mazes 5, 6, 8, 11, and 12, for children scoring greater than 1 SD below (FIG. 2A), or less than 1 SD below (FIG. 2B) the mean (mean=100; SD=15) on Basic Reading, Reading Comprehension and Broad Reading measures. The examples come from two participants enrolled in the same elementary school, scoring within a similar range on all measures (within 0.5 SD of the mean), except Reading Comprehension. In this case the reading impaired student scored 1.6 SD below the mean.

FIG. 3 depicts path analysis based on Pesky Efficiency Score generated from z-scores of time and distance traversed through the maze. Path taken by a typically developing child over all six trials of Maze 8 (typical reader). Path taken by a child with RD over all six trials of Maze 8 (atypical reader). Solid lines represent walls in the maze; dotted lines indicate the location of error zones. A negative z-score indicates high performance efficiency (−3.0, solid shapes) and positive z-scores refer to inefficient maze performance (+3.0, hollow shapes).

FIG. 4 is a flowchart depicting the go/no-go strategy for the development of the research tool. We have advanced the research program through the first phase of development demonstrating proof-of principle for the use of the maze as an early detection tool for reading ability in native and nonnative English speakers. We are expanding our participant pool in order to examine a sample which more closely resembles the population of the US. In addition we are developing the prediction algorithm for realtime analysis within the Web-based virtual maze tool.

FIG. 5 depicts how children with RD exhibit impaired performance on the virtual Hebb-Williams maze task. Children, ages 8-13 years, with RD (open circles) exhibit inefficient maze solving abilities compared to children without RD (filled circles), across six trials for Mazes 5, 6, 8, 11, and 12.

FIG. 6 shows how maze performance may be a better predictor of future reading ability than early reading measures. Graph (A) show the interaction between trial and reading ability on virtual maze performance in 5-6-year-old children (n=68). Statistically significant interaction between reading ability, maze, and trials on vHW maze performance was found (F_{15,975}=1.7, p<0.05). Struggling readers (atypical, n=14) exhibit inefficient performance on this virtual maze task when compared to typical (n=54) readers in this age group. Graph (B) depicts the results of a longitudinal study showing the interaction between trial and reading ability on virtual maze performance in 5-6-year-old children with reading ability examined two years later. Reading measure (WJ-III) for children 8 years of age have higher reliabilities, ranging from 0.93-0.95, when compared to reading measures in young children which range from 0.38-0.74. Children with RD exhibit impaired performance on the virtual Hebb-Williams maze task. Children, ages 8-13 years, with RD (open circles) exhibit inefficient maze solving abilities compared to children without RD (filled circles), across six trials for Mazes 5, 6, 8, 11, and 12. These data indicate that the maze task was able to identify more children as reading impaired than current early reading measures.

FIG. 7 depicts the interaction between maze and reading ability on virtual maze performance. Graph (A) shows the interaction between maze and reading ability on virtual maze performance in 5-6-year-old children. Statistically significant interaction between reading ability, maze and trials on vHW maze performance was found (F_{15,975}=1.7, p<0.05). Struggling readers (atypical, n=14) exhibit inefficient performance on this virtual maze task when compared to typical (n=54) readers in this age group. Graph (B) show the interaction between maze and reading ability (measured at age 8) on virtual maze performance in 5-6-year-old children. Atypical readers (open bars), identified based on performance on WJ-III, exhibit impaired performance across all maze configurations.

FIG. 8 illustrates further results using data related to other languages. Children 8-13 years of age were recruited from public schools in Bavaria, Germany or Idaho, US. All children (monolingual) were tested in their native language on reading measures (WJ-III in the US, SLRT-II and ELFE 1-6 in Germany). Children were identified as atypical readers based on reading scores 1 SD below the mean. Atypical readers showed similar learning curves whether native German-speaking (shallow orthography) or native English-speaking (deep orthography), suggesting language orthography does not influence performance on this task. Therefore this measure could be used as a nonlanguage based tool for the early identification of dyslexia regardless of language background. Taken together, this tool could be used to identify potential reading difficulties in language learners, such as English language learners, which are due to an intrinsic learning difference as opposed to a lack of sufficient exposure to spoken and written English.

### DETAILED DESCRIPTION OF THE INVENTION

The embodiments of the invention and the various features and advantages thereto are more fully explained with references to the nonlimiting embodiments and examples that are described and set forth in the following descriptions of those examples. Descriptions of well-known components and techniques may be omitted to avoid obscuring the invention. The examples used herein are intended merely to facilitate an understanding of ways in which the invention may be practiced and to further enable those skilled in the art to practice the invention. Accordingly, the examples and embodiments set forth herein should not be construed as limiting the scope of the invention, which is defined by the appended claims.

As used herein, terms such as “a,” “an,” and “the” include singular and plural referents unless the context clearly demands otherwise.

The Hebb-Williams (HW) maze is a closed-field maze which consists of twelve distinct configurations, with three measured levels of difficulty (47). This maze has been employed to examine learning and memory abilities in mice with neocortical malformations (48, 49), similar to those identified through postmortem analysis of humans with dyslexia (50), and mice with a mutation within the Dcdc2 gene (38). These studies have demonstrated that animal models of RD are impaired at this visuospatial learning task. Successful maze learning requires the subject to reach a goal within the least amount of time by applying previously learned knowledge. An advantage of this virtual environment is the ability to effectively model the experience of walking through a maze, which may more accurately parallel the experience across species.

In the laboratory, a virtual environment was successfully created for the Hebb-Williams mazes (vHW) that mimicked the physical version of the maze used in our laboratory to examine visuospatial abilities in animal models of reading disabilities. We used this virtual environment to compare human performance to mouse performance on a physical version of the maze. A cross-species (i.e., mouse-human) comparison demonstrated similar performance efficiencies on this task. In addition, we demonstrated that both 5-6-year-old and 8-13-year-old children with reading difficulties exhibited a similar impairment to animal models of the disorder (i.e., mice which were genetically altered to create a microdeletion in a candidate dyslexia susceptibility gene [CDSG]).

Our data thus far suggest that regardless of the nature of a child's reading impairment (e.g., specific reading impairment, reading comprehension, reading fluency), there are significant differences in their overall performance on the vHW maze task compared to typical readers. We theorize that the vHW maze task taps a number of underlying processes associated with reading, such as executive function, working memory, attentional control, and visual processing skills, and that developing a more thorough understanding of how the vHW maze taps these constructs will provide valuable insight into its use as an early screening for reading difficulties.

Accordingly, the vHW maze offers the potential to serve as a reliable, nonlanguage based predictor of reading difficulty which can improve our early identification and intervention efforts for young children, including native speakers and language learners. Unlike current measures of cognitive processing areas, the vHW maze can be administered in the classroom, providing valuable information about a child's learning processes that could inform instructional practice. Thus, the vHW maze can fill important gaps in the extant range of early identification screeners, and further our understanding of the cognitive processes associated with reading.

Need for the vHW maze task and limitations of current reading screeners. Given the complexity of reading, some researchers question whether early identification of reading impairment is practicable (Thompson, et al. 2015). For example, reading impairment at the word level is most often conceptualized as a weakness in phonology (Ferrer, et al.), and screening tools that tap phonological abilities have been shown to be useful in identifying children at risk for word level reading impairments. Phonological skills such as segmenting and blending are particularly useful predictors, as are skills related to rapid automatic naming (RAN) (Frijters, et al. 2011).

There are a number of assessments that reliably tap phonological and early reading constructs and are in wide use in schools. Examples include the Comprehensive Test of Phonological Processing (CTOPP-2), the Test of Word Reading Efficiency (TOWRE-2), the Texas Primary Reading Inventory (TPRI), and numerous others, including a variety of curriculum-based measures of reading. These measures assess phonological processes, word level reading abilities and fluency. There are fewer measures available that screen for early language impairments, which have been associated with reading comprehension. Although group level differences on measures that are consistently reported in the research, they have been found to be less accurate when used to make diagnostic decisions at the individual level (Adlof, et al. 2010; Pennington, et al. 2012).

Additionally, an increasing number of studies examining neurocognitive processes associated with reading indicate a variety of processes beyond phonological may be associated with specific types of reading impairment. For example, Bailey, Hoeft, About, & Cutting (2016) reported a distinct neural profile linked to domain-general abilities for students with specific comprehension impairment compared to both typical readers and to those with specific word reading impairment. A recent review of neurocognitive theories of reading concluded that there are different cognitive processes associated with different subtypes of specific word impairment (Hadzibeganovic, van den Noort, Bosch, Perc, van Kralingen, Mondt, & Coltheart 2010), including attentional control (Friedmann, et al. 2010; Rayner, et al. 1989), visual processing of letters and their positions within words (Friedmann & Rahamim 2007), and working memory and retrieval difficulties which impair building a large sight vocabulary for rapid and automatic recognition (Friedmann & Lukov 2008).

In summary, current reading screening tools typically focus on phonological and language based measures only, which is problematic in two main ways, including:
1. CURRENT PREDICTORS WORK WELL AT THE GROUP LEVEL, BUT LESS WELL AT THE INDIVIDUAL LEVEL, WHICH IS HOW STUDENTS ARE REFERRED FOR EVALUATION FOR READING DIFFICULTIES. Although there is strong research support for a “phonological core” deficit of reading difficulties (Shaywitz 1996), recent studies indicate that they may arise as a result of multiple and independent cognitive disorders (Bosse, Tainterrer, & Valdois 2007). This is verified by large-scale attempts to apply predictive models to classify individuals at-risk for reading impairment. For example, Adlof, et al. (2010) found that different combinations of predictor variables in kindergarten were required to optimally predict reading impairments in second grade versus those in eighth grade. Phonological measures were more predictive of second grade reading impairments, whereas language measures were more predictive of those encountered in later grades. Pennington, et al. (2012) found that multiple deficit models of reading impairment more accurately identified cases at the individual level than single deficit models. However, even the best fitting model in their study, which allowed for both single and multiple-predictor models, only accurately identified 46% of their sample of students with reading difficulties. Kibby, et al. (2014) reported that although phonological awareness and rapid automatized naming predicted every aspect of reading assessed, working memory and attentional control also contributed to the predictive model. Similarly Bosse, et al. (2007) found that visual processing accounted for unique variance after controlling for phonological processing skills. These findings suggest that there are other important predictors and that a multiple-risk framework of identification may lead to more accurate identification of the heterogeneous manifestations of reading impairment (Pennington, et al. 2012).
2. CURRENT READING SCREENING MEASURES RELY ON LANGUAGE-BASED MEASURES ONLY. Related to the first issue, most of the measures used to identify students with reading impairment involve a language or language-related task. A model of reading offered by Perfetti, et al. (2005), depicts the complex processes that are required for efficient reading and that are consistent with current understandings of the reading network. Included in the network is the need for executive function to orchestrate the process, attentional control, working memory and visual processing (e.g., visual attention, visuospatial abilities and spatial cuing) required to access print, and to map visual (orthographic) information onto auditory (phonological) and conceptual (semantic) representations (Pugh, et al. 2000). Screeners and diagnostic tools that tap additional components of the reading network may be useful in identifying students at risk for reading impairments. Recognizing that not only the final “score” is important, but it is the process of navigating the maze that can reveal aspects with regard to a student's visual attention, visuo-spatial ability, and spatial cueing.

In applying the system and methods of the present disclosure, we will code students' behaviors with time stamps, combining sequential behavioral codes into a textual strings then use tools such as Google Word2Vec (Mikolov & Dean 2013) to classify behaviors based on pattern similarity. This process helps us incorporate clustering results into predictive modeling. The patterns revealed will be crucial information for gaining insights regarding students with disparate abilities.

In our previous work, we have found that there are significant differences in performance on the vHW maze between children who are good readers and those who are reading impaired. In our studies, we defined reading impairment as performance on a cluster score of the Woodcock Reading Mastery Test (WRMT-3) or the Woodcock Johnson Reading Tests of Achievement (WJ-IV) was more than 1 SD below the mean. We used the cluster scores for Basic Reading Skill, Reading Comprehension and Reading Fluency. Our results indicated that performance on the vHW maze task was impaired for students with one or more reading scores at this level, regardless of the area of reading impairment (e.g., poor decoding, poor comprehension, or both). This suggests that the vHW may be a useful early predictor of reading difficulties among all forms of RD, not simply poor decoding or poor comprehension. Thus, the system and the methods utilizing the same, may be utilized as a generic test for determining the RD in children, regardless of the underlying reason for the RD.

The vHW maze may successfully tap into higher order processes as well. Solving the maze tasks requires that the individual to use both reference and working memory processes, and to develop a cognitive map of their environment. The notion that individuals create cognitive maps, or mental representations of their environments, has been discussed for decades. Tolman (1948) discussed the idea of two different types of cognitive maps that are utilized in optimal conditions (i.e., comprehensive map), or in suboptimal conditions, those situations which lead to “intense frustrations” (i.e., strip-map). Comprehensive maps take the entire environment into consideration to navigate in the most efficient manner, as opposed to the strip map, which reflects a narrower, single path, or turn-by-turn, style of decoding the situational task. Although the vHW maze clearly requires the utilization of visual and spatial processes which parallel those necessary in successful acquisition of reading, this task also requires higher-order executive processes for efficient navigation. All children successfully learn the task, regardless of difficulty, but the way in which they solve the mazes is fundamentally different. For example, children, 8-13 years of age, with reading impairment show reduced performance efficiency on this task regardless of their specific deficit. We believe that this vHW task represents a unique identification tool which will be successful in identifying individuals at risk of reading impairment regardless of their specific deficit. Further, examination of the approach and performance navigating the vHW maze environment may provide useful insight into the specific difficulties a child may be experiencing.

Use of the systems and methods described herein will contribute significantly to our ability to predict students, at an early developmental stage, who are at an increased risk for reading difficulties. The relationship between performance on the vHW maze task and reading ability in children may demonstrate that the virtual environment, which can be distributed via a Web-based platform and therefore accessed by anyone with internet access, may be a low-cost, time efficient, easy to use tool for the early detection of reading impairment. Additionally, the ease of administration, and future plans for automated scoring, will significantly reduce the training needed to accurately administer, or perhaps be fully automated with no need for specialized training. For example, we have invented a system for detecting dyslexia in children wherein the system comprises a set of virtual HW mazes. A child completes the maze using keyboard strokes and during the pendency of the progress, we collect data regarding the relative position of the curser/progress. The child completes several mazes, each several times. The data is then collected and scored as compared to a control. The score is then utilized to evaluate dyslexia or reading disorder wherein a score below the control by at least one standard deviation signifies a predisposition or occurrence of RD.

Therefore, applying the system as described herein, an individual child may perform the required number of vHW mazes wherein the data is collected and compared to the control and a score generated for the child, which can be utilized to indicate a presence or absence of RD in the child. Early detection of RD, therefore, can be treated with appropriate procedures to eliminate or reduce the impacts of RD on the child through his or her education.

### vHW MAZE SYSTEM

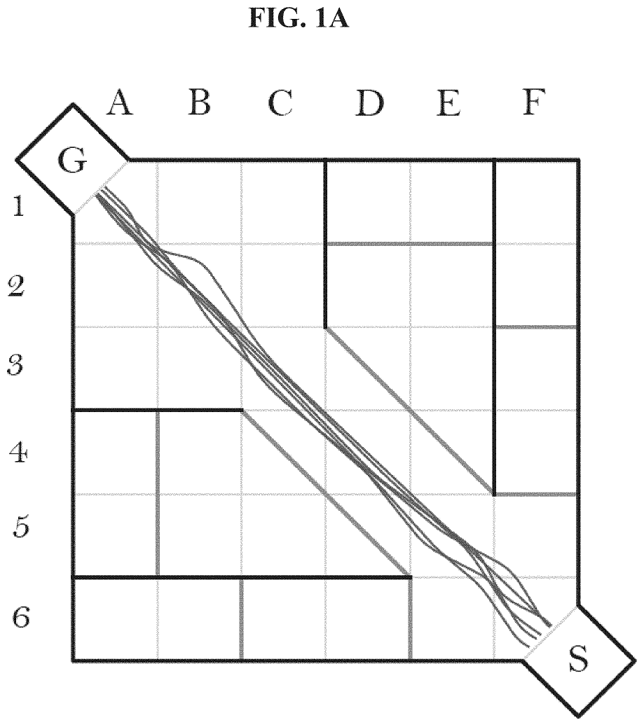

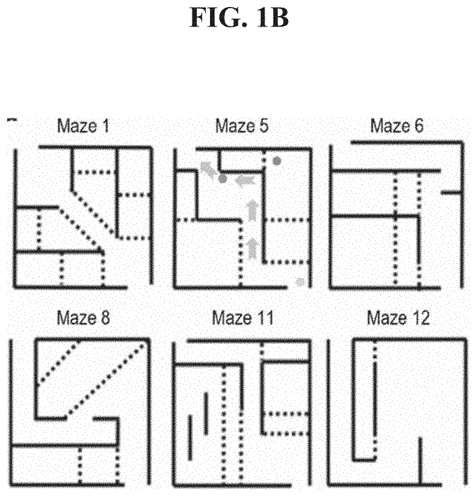

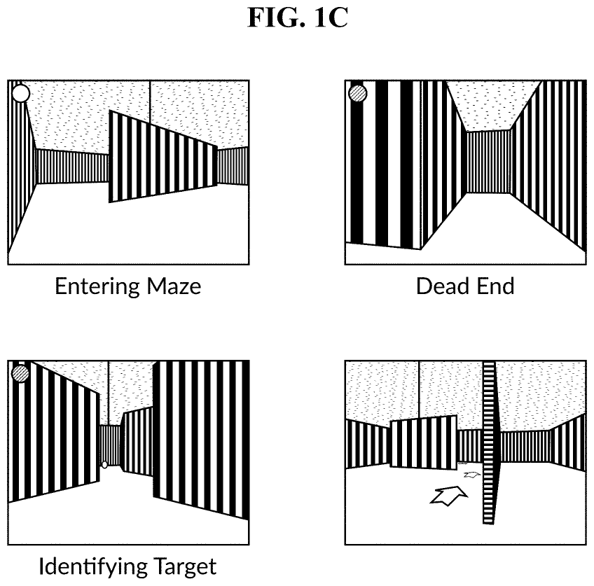

The virtual maze System comprises several vHW mazes (see FIG. 1) and analysis software were created using the Unreal Development Kit and Java, respectively. All of our experiments to date have been performed on either a Dell Latitude or an Alienware PC laptop. Mazes are displayed at a resolution of 1600×900 in full screen mode. The maze environment may be administered via a stored electronic copy on an infidel computing device, or, to make this assessment feasible for practitioners to administer, via a Web-based assessment tool that can be administered and automatically scored by anyone with an internet connection.

The mazes are configured to allow for different types of learning and memory processes which require different decoding skills. The training maze (Maze 1) is depicted in FIG. 1A. This maze, like all the mazes, is designed within a 6×6 grid (A1-F6), creating 36 individual cells. Each cell consists of 256 units (1 unit=1 cm), creating a 16×16 unit grid within each individual cell. This design allows us to examine the path taken by the individual at multiple levels (i.e., within an individual cell or down to a single unit within a cell). For this maze the interior walls create a series of corridors and dead ends. Walls are present along the border of the following cells: C1/D1, C2/D2, E1/F1, E2/F2, E3/F3, E4/F4, A3/A4, B3/B4, A5/A6, B5/B6, C5/C6, and D5/D6. The most efficient path in Maze 1 is to traverse F6, E5, D4, C3, B2, and A1 on the diagonal (along the diagonal of the square). If the individual takes the path to the right of the start box (F6, F5, F4, F3, F2, F1) they will come to a dead end, and will have committed two errors (crossing into cell F4 from F5, and crossing into F2 from F3). There are six other error zones indicated by the dark gray lines at positions D1 to E1, the diagonal of cells D3 to E4, the diagonal of cells C4 to D5, border of A4/B4 to A5/B5, the border of B6/C6, and D6/E6.

Maze 1 is used as a training maze because the participant can see the target from the start position. Once the individual has crossed into cell F6 a wall will block any reentry attempts into the start box. No sound or motion is detectable by the participant when this happens, it will simply be visible if and when the participant turns around. This is an example of a maze which requires reference learning and memory skills. The individual must remember that they need to remember to traverse a general straight path in order to efficiently solve the maze over the course of 6 trials. The path taken across all six trials by a single participant is depicted in the schematic by the think black lines extending from the start box (S) to the goal box (G).

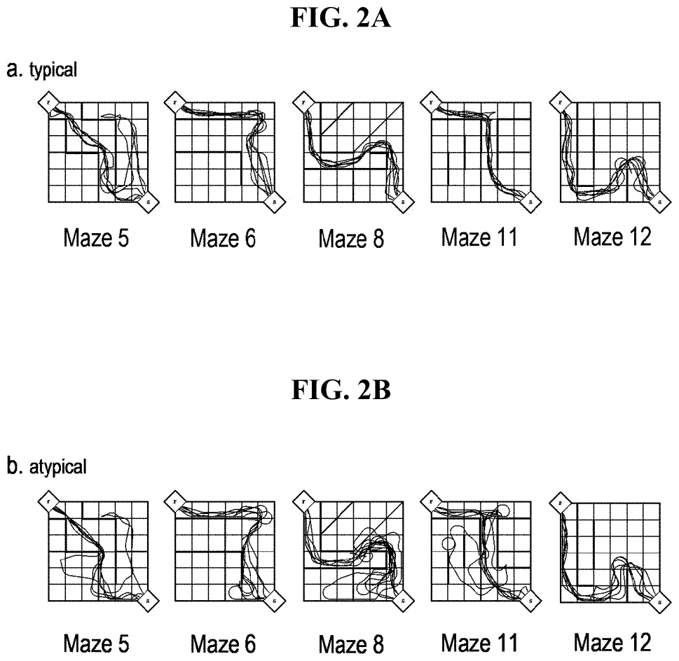

Other mazes, depicted in FIG. 1B may require that the individual follow a straight path, in general, to the target, or remember a series of correct turns to reach the goal box. Maze 1 in FIG. 1B requires that the individual remember a series of correct turns in order to successfully navigate the maze. This would require more working memory skills than reference memory skills. of FIG. 1A shows a schematic of the maze show a schematic of Mazes 1, 5, 6, 8, 11 and 12. The solid lines represent walls, while the dotted lines are error zones. Each maze is made up of 6×6 cells that make up the interior of the maze (see FIG. 2A, 6×6 grid depicted in light lines), similar to the grid of the physical maze. Each cell consists of 256 units (1 unit in Unreal is equivalent to ˜1 cm), with the goal and start boxes 300 units deep, and a viewing height (eye level) of 85 units from the ground. Participants navigate through the virtual environment, based on a first person perspective (PP1), at a constant velocity of 175 units/second and a maximum turn rate of 96 degrees/second.

The additional mazes, e.g., HW Mazes 5, 6, 8, 11, and 12 can be described as follows:

Maze 5, external walls, and internal walls from: A1/A2, A2/B2, A3/B3, B3/B4, C3/C4, B1/C1, C1/C2, D1/D2, C2/D2, C3/D3, C4/D4, C5/D5, with the most direct path from F6, E6, D6, D5, D4, D3, C2, B1, A1, or along the diagonal of squares from D3 toward A1.

Maze 6, external walls, and internal walls from A1/A2, B1/B2, C1/C2, D1/D2, E1/D2, F2/F3, and A3/A4, B3/B4, C3/C4, D3/D4, D4/E4, D5/E5. The most direct path is F6, F5, F4, F3, E3, E2, F2, F1, E1, D1, C1, B1, and A1. Again, traversal along a diagonal may be appropriate in certain circumstances.

Maze 8 has exterior walls and internal walls from A1/B1, A2/B2, A3/B3, B3/B4, A5/A4, B5/B4, C5/C4, D5/D4, E5/E4, E4/F4, and E3/E4. The shortest solution is F6, F5, F4, F3, E3, D3, D4, C4, B4, A4, A3, A2, and A1. Again, traversal along a diagonal may be appropriate in certain circumstances along the best path.

Maze 11 has exterior walls and internal walls at A1/A2, B1/B2, C1/C2, C2/D2, C3/D3, F1/F2, E1/E2, D2/E2, D3/E3, D4/F4, A4/B4, A5/B5, and B3/C3, B4, C4. The shortest solution is F6, E6, D6, D5, D4, D3, D2, D1, C1, B1, and A1.

Maze 12 has exterior walls and internal walls at A1/B1, A2/B2, A3/B3, A4B4, A5/B5, B5/B6, B2/C2, B3/C3, B4/C4, and D5/E5, D6/E6. The shortest solution is F6, F5, F4, D4, D4, C4, C5, C6, B6, A6, A5, A4, A3, A2, and A1. Again, traversal along a diagonal may be appropriate, the above solution uses whole squares. However, traversal from F6, E5, D4, C5, B6, A5, A4, A3, A2, and A1 is possible traversing exactly along the diagonal. Each of the above mazes is described for the shortest solution along whole blocks, though those of skill in the art will understand that traversal from a corner to another corner (along the diagonal of the square) is shorter.

To complete the vHW maze task, students are habituated to the virtual environment using the Maze 1 configuration (see FIG. 1A) and are told to “find the ball.” Once the student has acquired the target, a smiling cartoon star appears on the screen. Students are given a sticker following the completion of each trial that is placed on a sticker chart provided by the administrator. The sticker chart has proved to be a powerful motivator with children in this age group (5-13) and helps track their progress through the mazes. Students complete six consecutive trials with a 120 second maximum completion time per trial. If the student exceeds the maximum allowable time, floating arrows appear on the floor of the maze guiding the participant to the goal box; data is not collected once the maximum completion time has been reached. Students are randomly oriented within the start box at the beginning of each trial to simulate the experience of animal models completing the physical version of this task. Students navigate each maze a total of six times. A maze is completed when the participate finds the ball, or a maximum of 120 s has passed. Multiple trials are used to examine the learning rates across mazes for individuals with and without a specific reading impairment. A two minute distracter task, such as a video clip from an age-appropriate, popular children's movie, is used between each Maze to ensure that the participant does not try to utilize the same decoding strategy for consecutive mazes. A sample of screen shots is provided by FIG. 1B, showing entry to the maze, a dead end, identifying the target, and finally, arrows on the ground which are displayed in the event that a maximum of 120 s has been reached. The arrows are only displayed in the event the target is not identified in 120 s and is necessary to aid in the learning process. All students learn the task by the sixth trial. However, the learning rate and the decoding process differ between individuals with and without a specific reading impairment.

The visualization of each trial by a participant through the vHW maze is generated from data points captured at regular increments by the simulation software. The signal to capture information is generated at a hardware system level, thus time capture occurs using system resources for the best possible time resolution. These data points are pieced together using both Cartesian (x, y) coordinate and time information, producing a path drawing through a visualization of the virtual maze (FIGS. 2A and 2B). While the path drawings are constructed, both the amount of time spent and the number of samples associated with each grid location will be captured. Each maze consists of a six-by-six grid (depicted in light lines of the 6×6 grid). Each rectangular grid location is the finest granularity spatial positioning used in the original mazes. The solid black lines define the position of the walls; lighter lines indicate error zones. FIG. 2A demonstrates the paths taken (dark lines) over the six trials by an individual participant across different maze configurations. The data demonstrate that typical readers quickly learn the correct path and maintain that knowledge across the remaining trials. By comparison, FIG. 2B, the atypical readers often take more trials to find the target (i.e., ball/solution) and will often make errors in subsequent trials on the same maze, even after they have identified the correct path. These data demonstrate that typical readers learn the task quickly and maintain that knowledge for the remaining trials, whereas atypical readers take more trials to learn the task and often are unable to continue following the correct path in subsequent trials (i.e., more haphazard performance).

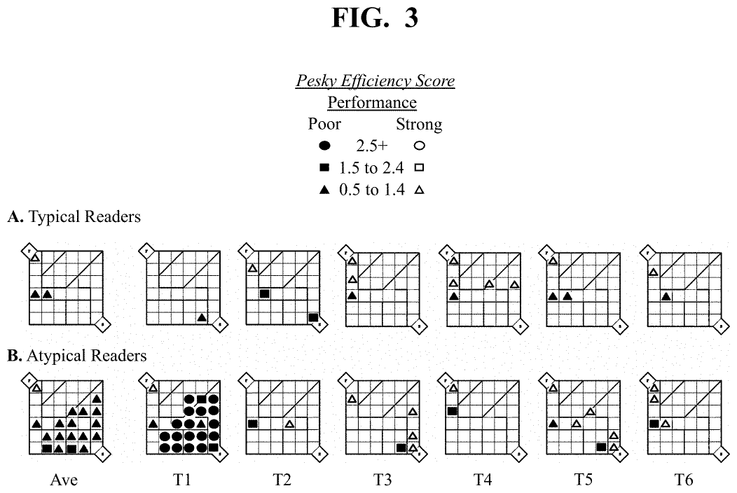

FIG. 3 depicts a composite standard value equally weighing time spent in a location and distance traveled within a position in the maze (Pesky Efficiency Score) is calculated and coded to represent a z-score value. Open shapes represent the least time/sample count (z-score=−3.0) and filled shapes represent the greatest (z-score≥+3.0). These maps provide us with a more dynamic representation of the problem solving strategies employed by participants (FIG. 3). Recently, we demonstrated that students with reading impairment show statistically different paths to acquire the target (ball or target), based on Pesky Efficiency Scores calculated for each maze position, compared to typical readers (Gabel, et al. 2016). The goal of the proposed project is to develop standards for performance that could be translated into criterion scores associated with reading difficulty risk at the individual level.

Two studies utilizing a virtual version of the Hebb-Williams maze task demonstrated that mice perform similarly to humans on this task (45, 46). Both typically developing mice and a mouse model Fragile X Syndrome (FXS) display similar performance efficiencies to their human counterparts (i.e., typically developing adults, and adults diagnosed with FXS, respectively) (45, 46). The vHW system, comprises a virtual environment for the Hebb-Williams mazes (FIG. 1) wherein the virtual environment mimicked the physical version of the maze utilized in our laboratory (1) to examine visuospatial learning abilities in animal models of RD (2). We used this virtual environment to compare human performance (18-23-year-olds [adults] and 8-13-year-olds [children]) to mouse performance on a physical version of the maze (1). A cross-species comparison demonstrated similar performance efficiencies on this task. In addition, we demonstrated that 8-13-year-old children with RD exhibited a similar impairment to animal models of the disorder (i.e., mice with a mutation within the Dcdc2 gene) (1).

Although the exact link between visuospatial learning on this task and reading ability has yet to be determined, research indicates that children and adults with dyslexia exhibit impaired visual processing and attention (3-7), and unsuccessful use of visual cues (8). Impaired visual processing and attention is thought to negatively impact a reader's graphemic parsing ability (9, 10), which requires both visual spatial attention skills and phonological skills (11). It has recently been reported that “multisensory sluggish attention shifting,” that is the integration at the word level of both visual (grapheme) and phonological (phoneme) processes, accounted for more than 30% of nonword reading performance of a sample of children with dyslexia after controlling for age, IQ, and phonological skills (12, 13). In this study we will examine participants' ability to successfully solve a series of virtual Hebb-Williams maze configurations. The completion of the task requires appropriate use of visual cues (i.e., based on the unique configuration of walls and alleys in each maze), working and reference memory abilities as the individual completes successive trials of each maze, which is dependent on visuospatial processing and attention (i.e., selecting the most relevant information) while navigating through the maze (14-17). These processes parallel those involved in reading, which may explain why both animal models of reading disorder, and children with impaired reading ability exhibit reduced performance on this task (1, 2). In addition, this virtual maze test does not require oral reporting (i.e., rapid access to phonological processing) or rely on text of any kind and therefore will not be influenced by a potential difference in reading experience between groups (18).

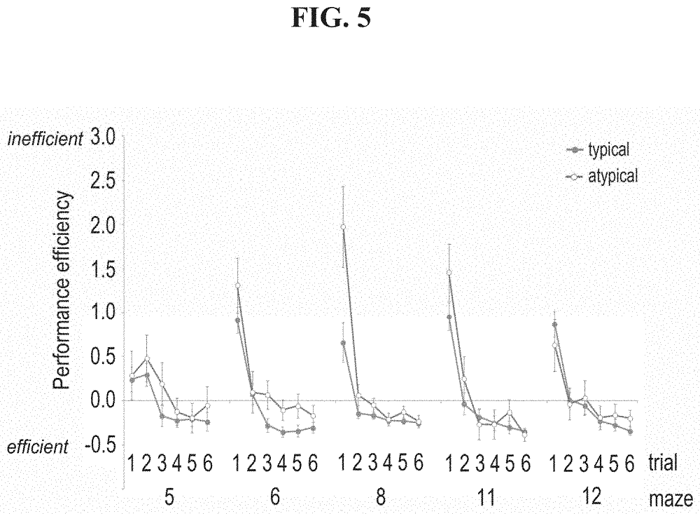

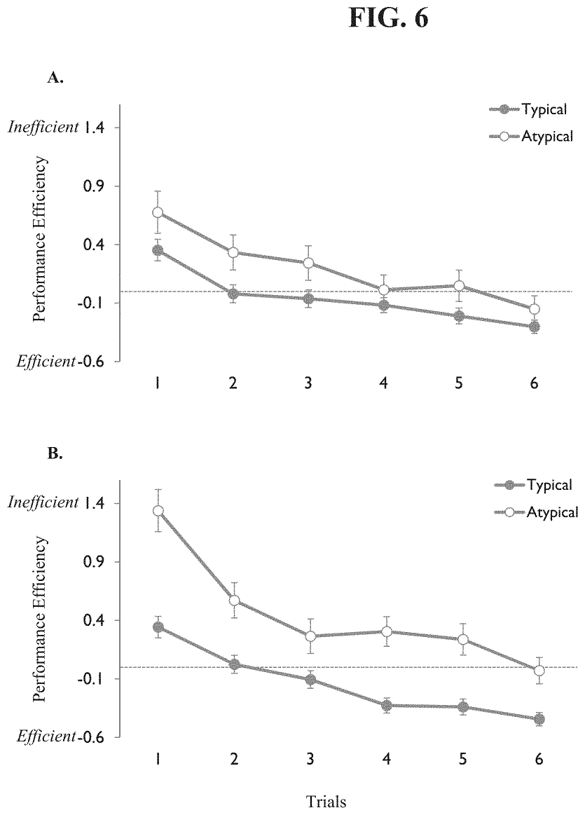

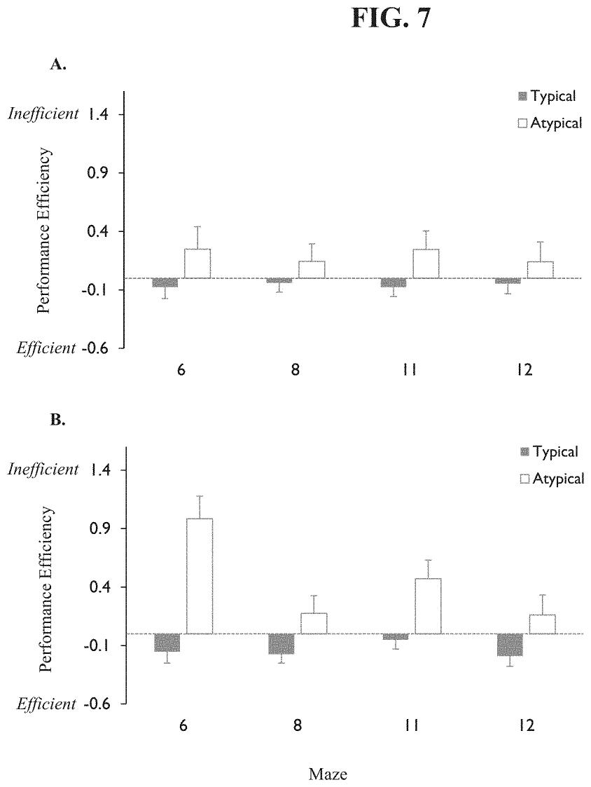

There is a strong scientific premise for this research based on our preliminary data translating the finding across species. A potential weakness is the possibility of a mixed population of participants (e.g., surface vs. phonological dyslexia; various CDSG risk variants). A mixed population would likely reduce the power of the study, however our recent work suggests that we are able to detect statistically significant and important differences between typical and atypical population among readers (FIG. 5) (1) and prereaders (FIGS. 6 and 7), despite a diverse population. By examining reading ability, phonological awareness, familial risk for dyslexia and specific CDSG risk variants we will ensure our ability to address our research question in a diverse sample, sharing common characteristics of RD, to produce impactful results.

The complete explanation of a complex neurodevelopmental disorder requires an understanding across multiple levels, including, but not limited to, cognition, behavior, and genetics. This study provides a unique framework for examining specific reading impairment from a multidisciplinary perspective to gain a better understanding of the factors which underlie this neurodevelopmental disorder. This study addresses several issues regarding possible confounds of examining sensory theories of dyslexia (18), such as utilizing a behavioral paradigm which does not rely on oral reporting or the use of letters; combining genetics (familial risk and presence of genetic risk variants of dyslexia), measures of reading and phonological awareness, with performance on the visuospatial learning task; as well as adding a longitudinal component which will enable us to determine how well prereading measures (cognitive, behavioral, and genetic) predict future reading performance.

From this one study, we will better understand the factors which influence the early detection of reading disorder. In addition, these studies will provide powerful insight into our ability to use the virtual Hebb-Williams maze as a suitable low-cost, time efficient, easy-to-use tool for early identification of specific reading impairment. Stronger understanding of the connections across learning deficits and reading ability can inform interventions that can be implemented earlier in a child's academic career. Early detection will help to close the gap between typically developing and reading-impaired children in acquiring reading skills. This will enable educators to employ early intervention measures in prereaders, as opposed to intensive remediation measures in later development, in order to significantly decrease the delayed acquisition of reading skills.

RESULT 1: We determine that altered visuospatial learning abilities on the virtual Hebb-Williams maze in children with RD are associated with CDSG genetic risk variants.

### INTRODUCTION

Studies examining virtual maze performance in children have only recently been reported. The few studies that have been published examining performance of children on virtual maze learning and memory tasks (19-24) have investigated small populations of participants (i.e., n≤10) per age group (20, 22). However, these studies have demonstrated that children as young as three years of age (22) are able to successfully navigate through maze environments similar to the Hebb-Williams maze. Unlike our virtual maze design, these studies used landmarks within the maze, textured interior walls, and extra-maze cues depicting a variety of environmental scenes. In this study we chose to model the virtual environment directly after the physical version of the maze employed in our laboratory. This design will ensure the greatest success in translating the results across species. Recent work from our laboratory suggests that 8-13-year-old children with reading impairment (i.e., cluster score≤85 on the Woodcock-Johnson III Test of Reading Achievement [WJ-III]) exhibit impaired performance on the vHW maze (1). The objective of this aim is to determine if performance on the vHW maze task correlates with the presence of known risk variants in CDSG.

For this study, we focused on two functional risk variants, READ1 and KIA-Hap, which we previously showed strongly interact synergistically to adversely affect several reading phenotypes (25, 26). READ1 is a highly polymorphic, purine-rich, compound short tandem repeat (STR) located in intron 2 of DCDC2, a known RD risk gene (27). We previously demonstrated that READ1 binds a human brain-expressed transcription factor called ETV6 with very high specificity and is capable of modulating reporter-gene expression from the DCDC2 promoter in an allele-specific manner (28). KIA-Hap is a three-marker risk haplotype encompassing the 5-prime half and upstream sequence of KIAA0319, a known RD risk gene consistently associated with lowered reading performance (29-32). Expression of KIAA0319 is lower in several cell lines that have this haplotype than in cell lines that do not (33). Additionally, expression from the KIAA0319 promoter in two human neural cell lines is reduced by the minor allele of a single nucleotide polymorphism (SNP) that resides in the KIAA0319 promoter and that is associated with this haplotype (34). Intriguingly, both of these functional risk variants, READ1 and KIA-Hap, are transcription control elements for two respective genes that strongly associate with RD, are encoded within 250 kb of each other in DYX2 (dyslexia associated locus number 2) on human chromosome 6p22, and appear to affect neuronal migration in early developing cortex (35, 36). Recent research demonstrates that KIAHap interacts with deleterious alleles of READ1 in a nonadditive manner to adversely affect reading and language performance, while protective alleles of READ1 epistatically negate the effect of KIAHap (25, 26).

We tested the working hypothesis that children with RD exhibit impaired performance on the vHW maze task and that this performance is correlated with specific READ1 and KIA-Hap risk variants. The rationale for this work is the need to understand the link between CDSG expression in cell lines and cognitive processing deficits reported in individuals with dyslexia to develop more comprehensive interventions to reach students with RD currently labeled as “nonresponders” to conventional methods. We expect that a primary outcome of this work will be the elucidation of the link between reading ability, genetics, and visuospatial learning.

### JUSTIFICATION AND FEASIBILITY

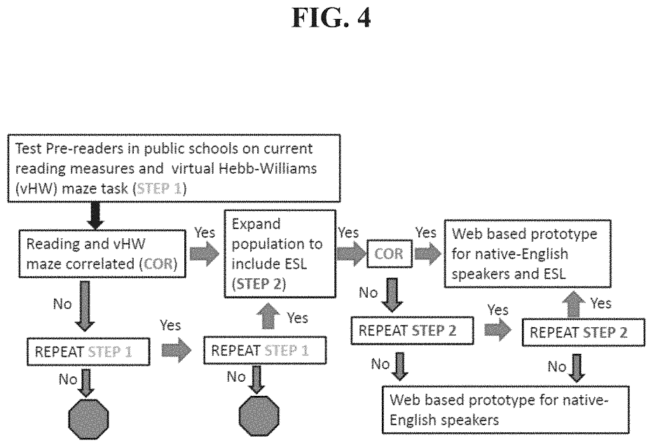

The objective of this study is to determine if performance on the vHW maze task correlates with the presence of known CDSG risk variants. We tested the working hypothesis that children with RD exhibit impaired performance on the vHW maze task (FIG. 2) and that this performance is correlated with specific READ1 and KIA-Hap risk variants. This hypothesis is supported by recent data collected from our joint research efforts and initial pilot data. The impaired visuospatial learning ability identified in children with a specific reading impairment parallels the deficit identified in animal models of the disorder (mice with genetically altered CDSG expression). Therefore, these data will enhance our understanding of the interplay between visuospatial learning deficits and the presence of genetic factors for children with specific reading impairment. We expect that a primary outcome of this work will be the elucidation of the link between reading ability, genetics, and visuospatial learning. These studies are essential in order to develop more comprehensive interventions for children not responding to conventional methods. In addition, these studies will provide powerful insight into our ability to use these tools as early detection methods for identifying children at risk for RD. The overall research strategy from initial data collection to implementation of a Web-based application for the early identification of reading disorder (RD) is outlined in FIG. 4.

Recent work from our laboratory demonstrates that adults (n=43, ages 18-23) and children (n=73, ages 8-13), exhibit similar problem solving abilities on the vHW maze to mice (n=12, 12-15 weeks postnatal) on the physical version of the maze. In addition, examination of 91 children (46 female) demonstrated that participants with reading impairment (12 male and 6 female) exhibited similar deficits on the vHW maze task (FIG. 2) as Dcdc2 knockout mice, a genetic animal model of reading disorder (2). These data demonstrate a statistically significant interaction between mazes examined, trials for each maze performed and reading group, F(20,1760)=1.76, p=0.02, effect size f=0.14. In addition, pilot work examining vHW maze performance and specific CDSG risk variants in 5-6-year-olds (prereaders) demonstrate Dcdc2 risk variants are associated with impaired performance on this task. Together these data constitute a feasibility test for the experiments proposed below.

### RESEARCH DESIGN

### Woodcock-Johnson IV Tests of Achievement (Reading)

The Woodcock-Johnson IV Tests of Achievement (WJ-IV) is a standardized, norm-referenced, individually administered battery of 22 tests designed to assess academic skill levels in several curricular areas (37). For this study, four reading subtests (Letter-Word Identification, Word Attack, Passage Comprehension and Reading Vocabulary) will be administered. Performance on Letter-Word Identification, in which a child names letters and reads words aloud from a list, and Word Attack, in which a child reads nonsense words aloud to test a child's phonetic knowledge, can be examined individually (reliabilities from 0.88-0.94) or combined as a Basic Reading Skills cluster (reliabilities 0.93-0.95) (38). For Passage Comprehension, students orally supply a missing word from a read passage. Reading Vocabulary requires participants to orally state synonyms and antonyms to given words. Passage Comprehension and Reading Vocabulary can be combined as a Reading Comprehension cluster with a similar increase in reliabilities. These subtests will be used to help determine areas of strength and weakness as related to the reading task. Scores for all subtests and clusters are reported as standard scores with a mean of 100 and a standard deviation of 15. Participants with basic reading scores below 85 on the WJ-IV will be operationally defined as reading impaired. Individuals who score <85 on Reading Comprehension, but not on measures of basic reading, are considered “poor comprehenders” and will not be grouped with individuals operationally defined as reading impaired (39, 40).

### Comprehensive Test of Phonological Processing-Second Edition (CTOPP-2)

The CTOPP-2 is a standardized, norm-referenced, and individually administered test used to identify individuals who exhibit impaired phonological abilities. Phonological awareness for this age group will be examined based on a composite score from three subtests, Elision, Blending Words, and Phoneme Isolation. Rapid Symbolic Naming will be assessed based on a composite score from two subtests, Rapid Digit Naming and Rapid Letter Naming. Raw subtest scores will be converted to standard scores (mean=100; SD=15), and then converted into composite scores. Participants with a Phonological Awareness score <85 will be operationally defined as reading impaired.

### Virtual Hebb-Williams Maze

The virtual maze is performed on an Alienware PC laptop during the studies described herein. Mazes will be displayed at a resolution of 1600×900 in full screen mode. There are 6×6 cells that make up the interior of the maze, similar to the grid of the physical maze. Each cell consists of 256 units, with the goal and start boxes 300 units deep, and a viewing height (eye level) of 85 units from the ground. Participants will navigate through the virtual environment at a constant velocity of 175 units/second and a maximum turn rate of 96 degrees/second using a Logitech Attack 3 joystick. A joystick will be used to decrease the influence of prior computer gaming experience in maze performance, since this type of joystick is rarely used in computer gaming today.

Participants will be habituated to the virtual environment using the Maze 1 configuration and the participants will be told to “find the ball.” Once the participant has acquired the target, a smiling cartoon star will appear on the screen. In addition, students will be given a sticker following the completion of each trial that will be placed on a sticker chart provided by the experimenter. This chart has proved to be a powerful motivator with children in this age group and helps track the participant's progress through the experiment. Participants will complete six consecutive trials with a 120 second maximum completion time per trial. If the participant exceeds the maximum allowable time, floating arrows will appear on the floor of the maze guiding the participant to the goal box (FIG. 1); data will not be collected once the maximum completion time has been reached. Participants will be randomly oriented within the start box at the beginning of each trial to simulate the experience of animal models completing the physical version of this task. Participants will complete Mazes 5, 6, 8, 11, and 12 in a single day of testing, and will be given a two minute distracter task (i.e., video clip from an age-appropriate, popular children's movie) in between each maze. All procedures are approved by Boise State University and Lafayette College IRBs.

### Path Analysis

The visualization of each trial run by a participant through the virtual maze is generated from data points captured at regular increments by the simulation software. The signal to capture information is generated at a hardware system level, thus time capture occurs using system resources for the best possible time resolution. These data points are pieced together using both Cartesian (x, y) coordinate and time information, producing a path drawing through a visualization of the virtual maze (FIG. 3). While the path drawings are constructed, both the amount of time spent and the number of samples associated with each grid location are captured. Each rectangular grid location is the finest granularity spatial positioning used in the original mazes and defines where the walls are positioned in the maze, with each maze consisting of a six-by-six grid of rectangles. A composite standard value equally weighing time spent in a location and distance traveled within a position in the maze (Pesky Efficiency Score) is calculated and color-coded to represent a z-score value. Open shapes represent the least time/sample count (z-score=−3.0), with ovals, squares, and triangles in order from least to greater time and filled shapes from triangle to square to oval represent the greatest (z-score≥+3.0). These maps will provide us with a more dynamic representation of the problem solving strategies employed by participants.

### Adult Reading History Questionnaire (ARHQ): Familial Risk

The ARHQ will be administered to the biological caregivers of the participants. The questionnaire is a reliable and valid screening tool for dyslexia in adults (41). The questionnaire consists of 23 self report items which are answered based on a 5-point Likert scale ranging from 0 to 4. The total score is divided by the maximum total score (score=92) to generate a percentile score ranging from 0 to 1. Higher scores are associated with greater reading difficulty. ARHQ scores will determine parental dyslexia and children will be classified as high or low familial risk using a 0.40 cutoff, which has a ˜80% sensitivity, specificity, and overall correct classification rate (41).

### Genetic Screening

Genetic screening procedures are approved by the IRBs at Boise State University, Lafayette College, and Yale University. For this study, we will focus on two functional risk variants, READ1 and KIA-Hap, which we previously showed strongly interact synergistically to adversely affect several reading phenotypes (25). READ1 is a highly polymorphic, purine-rich, compound short tandem repeat (STR) located in intron 2 of DCDC2, a known RD risk gene (27). We previously showed that READ1 binds a human brain-expressed transcription factor called ETV6 with very high specificity and is capable of modulating reporter-gene expression from the DCDC2 promoter in an allele-specific manner (28). KIA-Hap is a three-marker risk haplotype encompassing the 5-prime half and upstream sequence of KIAA0319, a known RD risk gene consistently associated with lowered reading performance (29-32). Expression of KIAA0319 is lower in several cell lines that have this haplotype than in cell lines that do not (33). Additionally, expression from the KIAA0319 promoter in two human neural cell lines is reduced by the minor allele of a SNP that resides in the KIAA0319 promoter and that is associated with this haplotype (34). Intriguingly, both of these functional risk variants, READ1 and KIA-Hap, are transcription control elements for two respective genes that both strongly associate with RD, are encoded within 250 kb of each other in DYX2 (dyslexia associated locus number 2) on human chromosome 6p22, and appear to effect neuronal migration in early developing cortex (35, 36).

### READ1 Genotyping

READ1 is genotyped by PCR amplification, purification of PCR products with ExoSAP-IT enzyme mix, and Sanger sequencing as previously described (25). Sanger sequencing will be performed at the Yale W. M. Keck DNA Sequencing Facility as per their standard sequencing protocol. Alleles will be called by an in-house C language program developed for this purpose (25).

### READ1 Microdeletion Genotyping

The 2,445 bp DCDC2 microdeletion encompasses the entire READ1 STR within its breakpoints, so it must be genotyped in addition to READ1 so that an accurate genotype can be achieved for apparent READ1 homozygotes (methods described in [25]). The microdeletion is genotyped by allele-specific PCR resolved on agarose-gel electrophoresis with the use of a three-primer reaction that generates a ˜600 bp amplicon from intact chromosomes and a ˜200 bp amplicon from chromosomes with the deletion, allowing heterozygotes and both homozygotes to be readily distinguishable from one another. PCR products are electrophoresed on 1% agarose gels with the use of standard 1×TBE buffer and ethidium bromide (0.2 mg/mL) via standard methods at 100-150 V, depending on gel size. Gels are imaged on a UV transilluminator and documented with a Bio-Rad Gel Doc XR imaging system. Genotypes are called from the gels manually.

### Statistical Analyses

Statistical tests will be performed using IBM SPSS Statistics 19 software (IBM Corporation, Somers, N.Y., US) or Microsoft Excel. A 2 (typical vs. atypical populations)×5 (Mazes 5, 6, 8, 11, 12)×3 (no risk factor, DCDC2 microdeletion, risk allele)×6 (trials 1-6) mixed factorial repeated measures ANOVA will be performed with maze and trial as the within-subjects factors. Errors and trial duration data will be collected and used to calculate performance efficiency. Performance efficiency is based on a calculation described by Shore, et al. (2001), which calculates the average of the standardized duration and error scores to provide a compound measure of performance on the Hebb-Williams maze (42). More specifically, performance efficiency is measured by computing the z-score (using the overall grand means and standard deviations from all subjects) for both error and time to complete the task. These two z-scores are averaged and both time and error are weighted equally. This provides a composite measure where large positive numbers reflect a relatively poor performance (i.e., longer duration and more errors than average). The dependent variable of performance efficiency on the Hebb-Williams maze task will be analyzed. Markers that deviate substantially from Hardy-Weinberg equilibrium or that have an overall call rate <85%, will be excluded from genetic analyses. Single marker SNP analyses of case-control status and quantitative traits will be performed with SNP and Variation Suite (SVS) v7.6.4 (Bozeman, Mont.). Haplotype-based association tests will be performed with KIAA-Hap haplotypes using PLINK v1.07 (43, 44). We will use a conservative Bonferroni correction to adjust for multiple testing.

### POTENTIAL PITFALLS AND ALTERNATIVE APPROACHES

Reading disability often includes a heterogeneous set of impairments (e.g., reading, language, and/or comprehension disabilities) so it is often challenging to establish parameters to identify a homogeneous sample. Of considerable debate is whether a there is a clear differentiation between individuals with specific language impairment (SLI) vs. specific reading impairment (39, 40, 45, 46). Three models have been postulated (i.e., severity, additional deficit, and the component model), of which only the component model describes “SLI only” as a group distinct from dyslexia (40, 47). Recent studies have suggested that the “SLI only” and “dyslexia only” group may be differentiated by measures of phonological skills vs. phonological representations (39), or based on differences in performance on measures of phonological awareness and verbal comprehension between the two groups (46). Based on our research design, and the continued debate of this topic, our operationally defined reading impaired group will likely contain individuals with SLI as well as specific reading impairment. However, we will be able to differentiate between “poor comprehenders,” reading impaired, and typical readers. In addition, a working hypothesis in our laboratory has been that different genetic variants may associate with specific deficits all of which underlie RD. If this hypothesis is correct, a sample of the population of individuals with RD could contain individuals with various CDSGs. If not all genetic variants are associated with a visuospatial learning deficit that can be identified with this task, then it may be difficult to demonstrate a difference in visuospatial ability. Our recently published findings suggest that reading impairment is associated with a visuospatial learning deficit on this task (1), despite the unlikely event we had a homogeneous population regarding risk variants of CDSGs. Recently published data suggests that individuals with RD, which have a microdeletion within DCDC2, show altered cortical connectivity (48-50) and visual processing deficit (51). KIAA0319, within 200 kb of DCDC2, has been shown to contribute to RD based on human, animal and cellular studies (27, 28, 31, 36, 52-55). In addition, the three marker risk haplotype in KIAA0319 (KIAHap) interacts with deleterious alleles of READ1 in a nonadditive manner to adversely affect reading and language performance, while protective alleles of READ1 epistatically negate the effect of KIAHap (25, 26). Therefore, we are confident that we will be able to detect a difference on this task between children with and without reading impairment and the genetic results will provide powerful insight into the link between genes, cognition and behavior. Familial risk assessment (i.e., ARHQ) will help determine genetic risk in individuals in the RD group who do not exhibit CDSG risk variants examined in this study. An additional area of Lastly, the use of rectangles in the path analysis follows from the original maze work (2), but given the granularity of the sample data, a much finer granularity heat map can be produced, providing a clearer picture of the captured data. Furthermore, other metrics can be applied to determine performance of individuals, such as, using amount of deviation from an optimal trajectory through the maze which would be generated automatically. These optimal paths would provide a base line measure for comparison of all participants and may be useful in Result 2 as we try to identify prereaders at risk for RD. Lastly, in this study gender will be analyzed as a covariate rather than a quasi-independent variable. In future studies, with a large enough sample, we will examine the quasi-independent variable of gender on our dependent measures.

### EXPECTED OUTCOMES

Based on preliminary examination of the similarities in performance between adults and children on this task, we anticipate that this approach will provide valuable information about visuospatial learning abilities in children with RD compared to typically developing participants. In addition, the link between CDSG risk variants and behavioral impairments on this task will further advance our knowledge on the link between these factors as we build upon recent research. These data will be invaluable as we examine performance in prereaders and will help us to understand the potential use of the virtual maze environment as a possible tool for the early identification of children at risk for RD.

RESULT 2: We determined that altered visuospatial learning abilities on the virtual Hebb-Williams maze in prereaders correlates with CDSG genetic risk variants.

### INTRODUCTION

As previously mentioned few studies have been designed to examine virtual maze performance in young children (19-22). However, children as young as three years of age (22) have been shown to successfully navigate through virtual maze environments. A pilot study conducted, in our lab, by Lafayette College students demonstrated that 5-6-year-olds are able to successfully navigate the virtual Hebb-Williams (vHW) mazes, and struggling readers were impaired at this task. The objective of this aim is to expand upon the pilot study to examine typical and struggling readers (i.e., based on reading readiness and phonological awareness) on the vHW maze task of visuospatial learning. We tested the working hypothesis that children at risk for RD will show similar visuospatial learning deficits on the vHW maze task to children identified as reading impaired (ages 8-13), and this will correlate with the presence of known CDSG risk variants. These data will enhance our understanding of the interplay between visuospatial learning deficits and the presence of genetic factors for RD. We expect that a primary outcome of this work will be the elucidation of the link between reading ability, genetics, and visuospatial learning in young children.

### JUSTIFICATION AND FEASIBILITY

As mentioned above, the long term goal of our research program is to identify children at risk for reading disorder, prior to acquiring reading skills. Through our joint collaboration, we have demonstrated the 8-13-year-old atypical readers exhibit poor visuospatial learning abilities on the vHW maze when compared to typical readers (1). The goal of this aim is to determine if prereaders at risk for RD, identified based on performance on the WRMT-III, CTOPP, and genetic screening for risk variants of CDSGs, will exhibit altered visuospatial learning abilities. These studies are essential in order to understand the link between CDSG expression in human cell lines and mice, cognitive processing deficits reported in individuals with dyslexia, and visuospatial learning behaviors in prereaders. In addition, these studies will provide powerful insight into our ability to use these tools as early detection methods for identifying children at risk for RD.

### PRELIMINARY STUDIES

We recently conducted a small pilot sample of 38 participants (20 male, 18 female) with 7 participants (3 male, 4 female) identified as struggling readers based on the WRMT-III test. An interaction between reading impairment, specific READ1 risk alleles, mazes (6, 8, 11, 12), and trials (1-6) (F_{15,480}=2.09, p<0.02, effect size f=0.25) was found. This pilot study provides preliminary evidence that the proposed experimental design described below is feasible.

### RESEARCH DESIGN

### Woodcock Reading Mastery Test (WRMT) III

The WRMT-III is a standardized, norm-referenced, individually administered battery of 9 tests designed to evaluate struggling readers, identify specific strengths and weaknesses in reading skills in order to plan targeted remediation, screen for reading readiness, and guide instructional decisions (56). For this study, the four reading subtests, Phonological Awareness, Letter Identification, Rapid Automatic Naming, Passage Comprehension, Word Comprehension and Listening Comprehension will be administered. The first three subtests produce a Reading Readiness Cluster, and the remaining three subtests provide an indication of the child's comprehension ability. Comprehension scores will provide us with a possible indicator as to whether children in our sample exhibit characteristics common of “poor comprehenders.” Scores for all subtests and clusters are reported as standard scores with a mean and standard deviation of 100±15. Participants with reading readiness cluster scores below 85 on the WRMT-III will be identified as struggling readers, all others as typically developed. Reliability coefficients of subtests range from 0.85-0.94; the average reliability for cluster scores is 0.95. Reading achievement test procedures are approved by the IRB at Boise State University.

### Comprehensive Test of Phonological Processing-Second Edition (CTOPP-2)

The CTOPP-2 is a standardized, norm-referenced, and individually administered test used to identify individuals who exhibit impaired phonological abilities (suitable for individuals aged 4-0 to 24-11). Phonological awareness for this age group will be examined based on a composite score from three subtests, Elision, Blending Words, and Sound Matching. Rapid Automatic Naming will be measured with the WRMT-III, so a Rapid Non-Symbolic Naming Composite Score will not be generated using the CTOPP-2. Raw subtest scores will be converted to standard scores (mean=100; SD=15) and then converted into composite scores. Participants who exhibit a composite score below 85 will be identified as struggling readers.

### Virtual Hebb-Williams Maze

Visuospatial learning will be examined using the same vHW task and procedures described in Result 1. In addition, Path Analysis procedures described in Result 1 will be employed. Based on pilot data, an additional training maze (Maze 5) will be utilized. For the younger age group, Maze 1 seems to familiarize the participant with the joystick, whereas Maze 5 acclimates the participants to the interior walls. Therefore, children will complete four mazes tasks (Mazes 6, 8, 11, and 12) with six trials per maze. A two-minute distractor task (i.e., an age-appropriate, popular animated movie) will be presented between each maze task. The joystick will be fixed to the table since younger children sit lower to the table causing them to push the joystick away from them, thus influencing their performance. All other parameters of the virtual environment and procedures were the same as adult populations. Hebb-Williams maze procedures are approved by the IRBs at Lafayette College and Boise State University.

### Adult Reading History Questionnaire (ARHQ): Familial Risk

As described in Result 1, the ARHQ will be administered to the biological caregivers of the participants, with percentile scores ranging from 0 to 1 (higher score associated with reading impairment). A percentile score of 0.40 will be used to predict parental dyslexia, which will indicate whether a child is a high (parental score≥0.40) or low (parental score≤0.40) familial risk.

### Genetic Screening

As described in Result 1, genetic screening procedures are approved by the IRBs at Boise State University, Lafayette College, and Yale University. As discussed in Result 1, we will focus on two functional risk variants, READ1 and KIA-Hap, which we previously showed strongly interact synergistically to adversely affect several reading phenotypes (25). Please see Result 1 for genotyping description.

### Statistical Analyses

Statistical tests will be performed using IBM SPSS Statistics 19 software (IBM Corporation, Somers, N.Y., US) or Microsoft Excel. A 2 (typical vs. atypical populations)×3 (no risk factor, DCDC2 microdeletion, risk allele)×4 (Mazes 6, 8, 11, 12)×6 (trials 1-6) mixed factorial repeated measures ANOVA will be performed with maze and trial as the within-subjects factors. Errors and trial duration data will be collected and used to calculate performance efficiency as described in Result 1. Heat maps will be developed, as described in Result 1, which will allow us to combine the path the participant took, with time, to get a more dynamic representation of the problem solving strategies employed by participants. Markers that deviate substantially from Hardy-Weinberg equilibrium or that have an overall call rate <85% will be excluded from genetic analyses. Single marker SNP analyses of case-control status and quantitative traits will be performed with SNP and Variation Suite (SVS) v7.6.4 (Bozeman, Mont.). Haplotype-based association tests will be performed with KIA-Hap haplotypes using PLINK v1.07 (43, 44). We will use a conservative Bonferroni correction to adjust for multiple testing.

### POTENTIAL PITFALLS AND ALTERNATIVE APPROACHES

Reading disability often includes a heterogeneous set of impairments so it is often challenging to establish parameters to identify a homogeneous sample (39, 40, 45, 46). We are be able to differentiate between typical readers, reading impaired individuals, and those described as “poor comprehenders,” however our reading impaired group will likely include individuals with SLI. In this study gender will be analyzed as a covariate rather than a quasi-independent variable, with future studies aimed at examining the quasi-independent variable of gender on our dependent measures. In addition, there is a possibility that we will not be able to correctly identify all individuals at risk for RD, since the WRMT-III is designed to identify struggling readers which may or may not lead to reading disability as the child develops. Genetic screening, including CDSG risk variants and familial risk, will increase the likelihood of capturing the majority of children at risk for RD. Result 3 is designed to determine the stability of our cognitive and behavioral findings over time and to examine which measurement(s) (i.e., early reading measures, vHW maze performance, genetic risk factors) is (are) more highly correlated with reading ability measured in 8-13-year-old children. Lastly, the vHW maze will only demonstrate a difference between those with and without a visuospatial learning deficit on this task, which may or may not be present in the broader population of individuals with RD. However, as previously discussed, specific variants of DCDC2 are associated with aberrant cortical connectivity (48-50) and visual processing deficits (51) in dyslexics. In addition KIAHap (risk haplotype in KIAA0319) interacts with deleterious alleles of READ1 (located within intron 2 of DCDC2) in a nonadditive manner to adversely affect reading and language performance (25, 26). Familial risk assessment (i.e., ARHQ) will help determine genetic risk in individuals in the RD group who do not exhibit CDSG risk variants examined in this study. In this study, we will be able to demonstrate whether specific READ1 alleles, which presumably alter DCDC2 expression, are associated with altered visuospatial learning on this task. Based on preliminary data, we are confident that we will be able to detect a difference on this task between typical and struggling readers, and the genetic results will provide powerful insight into the link between genetic risk, prereading measures and visuospatial learning. Together these tools provide an invaluable amount of data on subject performance across a measure of reading ability and visuospatial learning ability and, when combined with the genetic screening, will provide a wealth of information not currently available in a single study.

### EXPECTED OUTCOMES

Based on our results, we anticipate that struggling readers will be impaired at the vHW maze task and will correlate with specific CDSG risk variants. These studies will provide some clarity to the link between multiple factors which underlie RD. Examining these outcomes in young children are expected to have an important positive impact toward the early detection of RD. Early identification of children at risk for RD will lead to a significant decrease in the delayed acquisition of reading skills.

RESULT 3: We determined that the virtual Hebb-Williams maze is a better predictor than early reading measures based on a two-year longitudinal study of reading and maze performance.

### INTRODUCTION

We demonstrated the link between reading ability, performance on the vHW maze and specific genetic risk variants for reading disorder. Our initial data analysis suggests a link between reading, visuospatial learning and CDSGs, however these results relied on reading assessments administered to children at age 5-6. The predictive validity of the WRMT-III results with later reading ability vary from as low as 0.38 to as high as 0.74. In other words, children who present as at risk at a young age may develop strong reading skills over time, and vice versa. Sensory impairments can change over the course of development (i.e., experience) leading to a change in behavior (64). Therefore, it is essential that we reexamine prereaders when the participants are ≥8 years of age. We tested the working hypothesis that a proportion of children who tested as struggling readers at age 5-6 exhibit a reading impairment at age 8-9, which correlated with vHW maze performance (FIGS. 6 and 7), and genetic risk for reading disorder. In FIGS. 6 and 7 we show that maze performance may be a better predictor of future reading ability than early reading measures. Examination of virtual maze performance in 5-6-year old children (n=68) demonstrate a statistically significant interaction between reading ability, maze and trials on vHW maze performance (F_{15,975}=1.7, p<0.05). Struggling readers (atypical, n=14) exhibit inefficient performance on this virtual maze task when compared to typical (n=54) readers in this age group (FIG. 6A interaction between trial and reading ability; FIG. 7A interaction between maze and reading ability). Examination of the virtual maze performance based on reading designation two years later (age 8), when reading measure more accurately assess reading ability, demonstrate an even stronger interaction between trial and reading ability on virtual maze performance (FIG. 6B), and between maze and reading ability (FIG. 7B). Reading measure (WJ-III) for children 8 years of age have higher reliabilities, ranging from 0.93-0.95, when compared to reading measures in young children which range from 0.38-0.74. Children with RD exhibit impaired performance on the virtual Hebb-Williams maze task. Taken together these data indicate that the maze task was able to identify more children as reading impaired than current early reading measures. The rationale for this work is that these data enable us to determine the consistency of our cognitive and behavioral findings over time and to examine which measurement is more highly correlated with reading ability. We expect that a primary outcome of this work will be to provide valuable insight into a link between reading ability, visuospatial learning, and genetic markers of RD in readers and prereaders.

These data provide powerful information toward the development of this cost effective, time efficient, easy-to-use tool that may be used at very young ages for early identification of children at risk for reading disorder.

### PRELIMINARY STUDIES

As previously described, examination of 91 children (8-13 years of age, 46 female) demonstrated that participants with reading impairment (12 male and 6 female) exhibited impaired performance on the vHW maze. This data combined with pilot work examining vHW maze performance and CDSG risk variants in 5-6-year-olds (prereaders) demonstrate that specific DCDC2 risk variants are associated with impaired performance on this task. Together these data constitute a feasibility test for the experiments proposed below.

### RESEARCH DESIGN

### Woodcock-Johnson IV Tests of Achievement (Reading)

As described above, four reading subtests (Letter-Word Identification, Word Attack, Passage Comprehension and Reading Vocabulary) from the WJ-IV will be used. Performance on Letter-Word Identification can be examined individually (reliabilities from 0.88-0.94) or combined for as Basic Reading Skills Cluster Score (reliabilities 0.93-0.95) (38). Passage Comprehension and Reading Vocabulary can be combined as a Reading Comprehension cluster with a similar increase in reliabilities. These subtests will be used to help determine areas of strength and weakness as related to the reading task. Scores for all subtests and clusters are reported as standard scores with a mean of 100 and a standard deviation of 15. Participants with basic reading scores below 85 will be operationally defined as reading impaired. Individuals who score <85 on Reading Comprehension, but not on measures of basic reading or phonological abilities, are considered “poor comprehenders” and will not be grouped with individuals operationally defined as reading impaired (39, 40).

### Comprehensive Test of Phonological Processing, Second Edition (CTOPP-2)

As described above, the CTOPP-2 will be used to identify individuals who exhibit impaired phonological abilities. Phonological awareness for this age group will be examined based on a composite score from three subtests, Elision, Blending Words, and Phoneme Isolation. Rapid Symbolic Naming will be assessed based on a composite score from two subtests, Rapid Digit Naming and Rapid Letter Naming. Raw subtest scores will be converted to standard scores (mean=100; SD=15), and then converted into composite scores. Participants with scores <85 will be operationally defined as reading impaired.

### Virtual Hebb-Williams Maze

The virtual Hebb-Williams (vHW) maze experiments will be conducted as described in Result 1 with a few modifications. Since we are retesting children at a later stage in their development, we do not want to have the participants complete the exact same subset of tests. Briefly, participants will still be habituated to the virtual environment using the Maze 1 configuration, given the same instructions to “find the ball,” and provided with the same behavioral reinforcement. However, performance will be measured on Mazes 6, 8, 9, and 10. This will allow us to examine consistency in performance over time (Mazes 6 and 8 will be performed at both time points), as well as acquire additional data on maze configurations (Mazes 9 and 10) that show comparable learning behaviors across species (42). As previously described, participants will complete six consecutive trials with a 120 second maximum completion time per trial. If the participant exceeds the maximum allowable time floating arrows will appear on the floor of the maze guiding the participant to the goal box; data will not be collected once the time allotment has been reached. Participants will complete the selected mazes in a single day of testing and will be given a two minute distracter task (i.e., video clip from an age-appropriate, popular children's movie) in between each maze. All procedures are approved by Boise State University and Lafayette College IRBs.

### Path Analysis

As described above, the visualization of each trial run by a participant through the virtual maze is generated from data points captured at regular increments by the simulation software. These data points are pieced together using both Cartesian (x, y) coordinate and time information, producing a path drawing through a visualization of the virtual maze. These maps will provide us with a more dynamic representation of the problem solving strategies employed by participants and will demonstrate how those strategies may change over time.

### Genetic Screening

Results from previously performed genetic screening analysis will be incorporated into this study. No additional genetic samples will be collected or analyzed.

### Statistical Analyses

Statistical tests will be performed using IBM SPSS Statistics 19 software (IBM Corporation, Somers, N.Y., US) or Microsoft Excel. A 2 (typical vs. atypical reader)×3 (no risk factor, DCDC2 microdeletion, risk allele)×4 (maze)×6 (trials 1-6) mixed factorial repeated measures ANOVA will be performed with maze and trial as the within-subjects factors. Errors and trial duration data will be collected and used to calculate performance efficiency as described in Result 1. Heat maps will be develop as described in Result 1, which will allow us to combine the path the participant took, with time, to get a more dynamic representation of the problem solving strategies employed by participants. Multiple regression analyses will be used to investigate whether prereading measures, performance on the vHW maze task, and genetic risk correlates with reading ability in children 8-13 years of age.

### POTENTIAL PITFALLS AND ALTERNATIVE APPROACHES

Potential pitfalls in regard to the influence of gender on visuospatial learning ability and genetic variability within our sample are similar to those discussed above. In future studies, with a large enough sample, we will examine the quasi-independent variable of gender on our dependent measures. Furthermore, other metrics can be applied to determine which factors best predict future reading ability. For example, as opposed to examining performance across trials, the performance efficiency z scores can be converted to T scores, so the natural log of the values can be taken and the slope of this decay function of the learning curve can be calculated. A multiple regression analysis can be performed to examine reading measures, visuospatial learning based on slope, and presence of specific genetic risk variants as predictors of reading ability. Lastly, based on a priori power analysis using the effect size from data collected in our laboratory, we will need ten subjects within the atypical reading group, with as many falling within each genetic group (no risk variant, DCDC2 microdeletion, risk allele for reading disorder). To date, the populations we have been working with exhibit an 18% rate for reading impairment. Therefore, we would need approximately 166 overall participants for this study. Despite attrition rates in longitudinal studies, we will have a large enough sample size to demonstrate statistically significant interaction between these variables.

### EXPECTED OUTCOMES

Based on recent data and previous reports published within the field, we expect to determine the predictive validity of vHW performance at ages 5-6, with later reading ability assessed at ages ≥8. These data provide new information regarding the stability of visuospatial learning abilities across important stages of reading development in children with and without reading impairment. Furthermore, we will be able to clarify the relationship between cognitive measure of reading ability, visuospatial learning, and specific CDSG risk variants. Taken together, these studies will help us to understand the potential use of the virtual maze environment as a possible tool for the early identification of children at risk for RD.

### FUTURE DIRECTIONS

### Examination of the Influence of Gender on Maze Performance

Previously published data suggest that gender may play a role in visuospatial learning (42, 57). Our previous research did not contain a large enough sample to examine gender as a quasi-independent variable, but analysis of gender as a covariate suggests there is a difference in performance between males and females on this task. In future studies, we will further investigate the role of gender in vHW maze performance.

### Examining Virtual Maze Performance and Genetic Risk Variants in Nonnative English Speaking Children

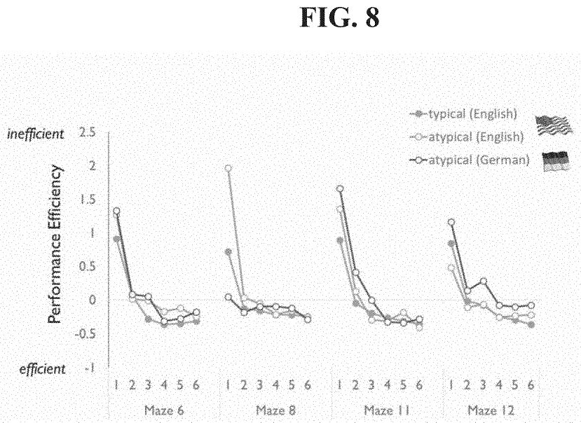

Recent research findings indicate a higher incidence rate of phonological dyslexia in children who speak English, a language with a nontransparent orthography, as opposed to surface dyslexia typically identified in children from countries with transparent orthographies (e.g., German). Dr. Gabel completed a Humboldt Fellowship for Experienced Researchers in Erlangen, Germany to determine if children who are native German-speakers exhibit a similar impairment on the vHW maze task compared to English-speaking children (nontransparent). The data collected from ELL, native German speakers, and multilingual children from a variety of language orthography backgrounds, demonstrate that virtual maze tool is able to identify children at risk of dyslexia (FIG. 8). Briefly, children (8-13 years of age) were recruited from public schools in Bavaria, Germany or Idaho, US. All children (monolingual) were tested in their native language on reading measures (WJ-III in the US, SLRT-II and ELFE 1-6 in Germany). Children were identified as atypical readers based on reading scores 1 SD below the mean (FIG. 8). Atypical readers showed similar learning curves whether native German-speaking (shallow orthography) or native English-speaking (deep orthography), suggesting language orthography does not influence performance on this task. Therefore this measure could be used as a nonlanguage based tool for the early identification of dyslexia regardless of language background. Taken together, this tool could be used to identify potential reading difficulties in language learners, such as English language learners, which are due to an intrinsic learning difference as opposed to a lack of sufficient exposure to spoken and written English.

This research, combined with the examination of genetic risk variants of RD, will advance our understanding of the biological basis of dyslexia.

Therefore, the development of the vHW mazes provides a novel opportunity to utilize spatial relations to detect and assess RD in children. The system comprises a set of vHW mazes which are run on a computing machine capable of tracking the position of the cursor as an infidel passes through each maze. The system tracks the particular coordinates along both time and Cartesian position and generates a scored based upon that performance.

While a number of HW mazes exist, data from the studies has identified that specific mazes provide for a greater predictive nature than others. Indeed, for example, Maze 1 is utilized to train someone how to perform the maze. Additional mazes can then be utilized to train how to seek for and find the exit. After training, the individual then completes at least four mazes, each being completed six times, for a total of at least 24 completions after the training. These totals are scored and tabulated and can be compared to a control.

Our data concludes that children with a RD fall below the control by at least one standard deviation using the preselected vHW mazes as described in the invention herein.

Therefore, methods of determine RD comprise the following steps: creating a vHW set of mazes comprising at least one training maze and at least four completion mazes wherein each completion maze is completed six times for a total of at least 24 completions. Scoring the relative position and time of the cursor during the pendency of each completion. Generating a score corresponding to the relative position and time of the cursor over the duration of the 24 completions.

The method may be further predictive by including a predictive step wherein a score of more than one standard deviation below the control indicates RD. Treating a patient for RD wherein the score is more than one standard deviation below the control.

For example, a preferred method comprises: a method of detecting reading disability (RD) in a child comprising administering to said child a set of at least four Hebb-Williams virtual mazes; wherein each maze is completed a total of six times, each completion taking no more than 120 seconds; and after completion of each maze, a gap of about 120 seconds between a maze and a subsequent maze; annotating the position of a cursor according to a coordinate system and time, calculating a score for the child for each completed maze, and wherein a calculated score can be compared to a predetermined control score to predict the presence of a reading disorder in said child.

Preferable, in the method a detection of a score more than one standard deviation below a control indicates a reading disorder wherein the child is treated for a reading disorder.

Preferably, in the method wherein each Hebb-Williams virtual maze is a 6×6 grid having a total of 36 cells, said cells arranged according to a Cartesian plot of A-F in the x-axis, 1-6 in the y-axis, and vertical walls in the z-axis. As indicated above, preferred methods include wherein each cell comprises 256 units, each unit having a length of 1 cm.

Our studies indicate that a time of no more than 120 seconds is preferable. Accordingly, the preferred methods including a limitation wherein a virtual path through the 6×6 grid takes no more than 120 seconds. If, in such 120 seconds, the goal is not completed, a lighted set of arrows indicates the path to the goal.

Preferably, a method comprise wherein a first Hebb-Williams virtual maze is Hebb-Williams Maze 1, wherein a child is oriented to starting from a start and finishing at a finish in Maze 1. Subsequently, after orienting the child, on Maze 1, the child is tested on at least four of Hebb-Williams Mazes 5, 6, 8, 11, and 12.

Hebb-Williams Mazes 1, 5, 6, 8, 11, and 12 are identified in detail herein, and have the following coordinates and solutions: Mazes 5, 6, 8, 11, and 12 have the following internal walls and most efficient solutions: Maze 5, external walls, and internal walls from: A1/A2, A2/B2, A3/B3, B3/B4, C3/C4, B1/C1, C1/C2, D1/D2, C2/D2, and C3/D3, C4/D4, C5/D5, with the most direct path from F6, E6, D5, D4, C3, B2, A1; Maze 6, and internal walls from A1/A2, B1/B2, C1/C2, D1/D2, E1/D2, F2/F3, and A3/A4, B3/B4, C3/C4, D3/D4, D4/E4, D5/E5 with the most direct path is F6, F5, F4, F3, E2, F1, E1, D1, C1, B1, and A1; Maze 8 having internal walls from A1/B1, A2/B2, A3/B3, B3/B4, A5/A4, B5/B4, C5/C4, D5/D4, E5/E4, E4/F4, and E3/E4; with the shortest solution is F6, F5, F4, E3, D4, C4, B4, A3, A2, and A1; Maze 11 internal walls at A1/A2, B1/B2, C1/C2, C2/D2, C3/D3, F1/F2, E1/E2, D2/E2, D3/E3, D4/F4, A4/B4, A5/B5, and B3/C3, B4, C4; with the shortest solution is F6, E5, D4, D3, D2, C1, B1, and A1; and Maze 12 with internal walls at A1/B1, A2/B2, A3/B3, A4B4, A5/B5, B5/B6, B2/C2, B3/C3, B4/C4, and D5/E5, D6/E6; and the shortest solution is from F6, E5, D4, C5, B6, A5, A4, A3, A2, and A1.

In a preferred embodiment, a virtual reading disability quantification system comprising a set of six mazes, a first maze indicated for training, each maze having exterior walls and internal walls as delineated within the exterior of a 6×6 grid with a cartesian plot of A-F along the x-axis and 1-6 along the y-axis and walls extending vertically in the z-axis. Maze 1 comprises internal walls along the border of the following cells: C1/D1, C2/D2, E1/F1, E2/F2, E3/F3, E4/F4, A3/A4, B3/B4, A5/A6, B5/B6, C5/C6, and D5/D6; with a start at F6 and a finish at A1, having the most efficient path to traverse F6, E5, D4, C3, B2, and A1 on the diagonal; and four additional mazes selected from the group consisting of: Maze 5, external walls, and internal walls from: A1/A2, A2/B2, A3/B3, B3/B4, C3/C4, B1/C1, C1/C2, D1/D2, C2/D2, C3/D3, C4/D4, C5/D5, with the most direct path from F6, E6, D5, D4, C3, B2, A1; Maze 6, and internal walls from A1/A2, B1/B2, C1/C2, D1/D2, E1/D2, F2/F3, and A3/A4, B3/B4, C3/C4, D3/D4, D4/E4, D5/E5 with the most direct path is F6, F5, F4, F3, E2, F1, E1, D1, C1, B1, and A1; Maze 8 having internal walls from A1/B1, A2/B2, A3/B3, B3/B4, A5/A4, B5/B4, C5/C4, D5/D4, E5/E4, E4/F4, and E3/E4; with the shortest solution is F6, F5, F4, E3, D4, C4, B4, A3, A2, and A1; Maze 11 internal walls at A1/A2, B1/B2, C1/C2, C2/D2, C3/D3, F1/F2, E1/E2, D2/E2, D3/E3, D4/F4, A4/B4, A5/B5, and B3/C3, B4, C4; with the shortest solution is F6, E5, D4, D3, D2, C1, B1, and A1; and Maze 12 with internal walls at A1/B1, A2/B2, A3/B3, A4B4, A5/B5, B5/B6, B2/C2, B3/C3, B4/C4, and D5/E5, D6/E6; and the shortest solution is from F6, E5, D4, C5, B6, A5, A4, A3, A2, and A1.

These systems and methods therefore may be utilized to predict RD in a patient and provides for early detection so as to induce treatment at an early age. Furthermore, the vHW mazes and the systems and methods described herein are generic as to the type of RD exhibited by the child and are also suitable for children having any primary language. Accordingly, children having a background in any of the language families can utilize the same methods and systems described herein to identify RD.

The systems and methods described herein may also be identified as a game for children wherein the game is predictive of RD in the child.

### REFERENCES

1. L. A. Gabel et al., Translating dyslexia across species. Annals of Dyslexia 66, 319-336 (2016).
2. L. A. Gabel et al., Mutation of the dyslexia-associated gene Dcdc2 impairs LTM and visuo-spatial performance in mice. Genes Brain Behav 10, 868-875 (2011).
3. A. Facoetti, M. Molteni, The gradient of visual attention in developmental dyslexia. Neuropsychologia 39, 352-357 (2001).
4. S. Gori, A. Facoetti, How the visual aspects can be crucial in reading acquisition? The intriguing case of crowding and developmental dyslexia. J Vis 15, 15.11.18 (2015).
5. R. Sireteanu, R. Goertz, I. Bachert, T. Wandert, Children with developmental dyslexia show a left visual “minineglect”. Vision Res 45, 3075-3082 (2005).
6. R. Sireteanu et al., Impaired serial visual search in children with developmental dyslexia. Ann N Y Acad Sci 1145, 199-211 (2008).
7. D. Menghini, A. Finzi, G. A. Carlesimo, S. Vicari, Working Memory Impairment in Children With Developmental Dyslexia: Is it Just a Phonological Deficity? Dev Neuropsychol 36, 199-213 (2011).
8. E. Moores, R. Cassim, J. B. Talcott, Adults with dyslexia exhibit large effects of crowding, increased dependence on cues, and detrimental effects of distractors in visual search tasks. Neuropsychologia 49, 3881-3890 (2011).
9. A. Facoetti, N. Corradi, M. Ruffino, S. Gori, M. Zorzi, Visual spatial attention and speech segmentation are both impaired in preschoolers at familial risk for developmental dyslexia. Dyslexia 16, 226-239 (2010).
10. C. Perry, J. C. Ziegler, M. Zorzi, Nested incremental modeling in the development of computational theories: the CDP+ model of reading aloud. Psychol Rev 114, 273-315 (2007).
11. A. Facoetti et al., The relationship between visuo-spatial attention and nonword reading in developmental dyslexia. Cogn Neuropsychol 23, 841-855 (2006).
12. A. Facoetti et al., Multisensory spatial attention deficits are predictive of phonological decoding skills in developmental dyslexia. J Cogn Neurosci 22, 1011-1025 (2010).
13. M. B. Krause, Pay Attention!: Sluggish Multisensory Attentional Shifting as a Core Deficit in Developmental Dyslexia. Dyslexia 21, 285-303 (2015).
14. J. McConnell, J. G. Quinn, Interference in visual working memory. Q J Exp Psychol A 53, 53-67 (2000).
15. E. J. Ploran, J. Bevitt, J. Oshiro, R. Parasuraman, J. C. Thompson, Self-motivated visual scanning predicts flexible navigation in a virtual environment. Front Hum Neurosci 7, 892 (2014).
16. O. Baumann, A. J. Skilleter, J. B. Mattingley, Short-term memory maintenance of object locations during active navigation: which working memory subsystem is essential? PLoS One 6, e19707 (2011).
17. K. C. Klauer, Z. Zhao, Double dissociations in visual and spatial short-term memory. J Exp Psychol Gen 133, 355-381 (2004).
18. U. Goswami, Sensory theories of developmental dyslexia: three challenges for research. Nat Rev Neurosci 16, 43-54 (2015).
19. E. K. Farran, Y. Courbois, J. Van Herwegen, A. G. Cruickshank, M. Blades, Colour as an environmental cue when learning a route in a virtual environment: typical and atypical development. Res Dev Disabil 33, 900-908 (2012).
20. E. K. Farran, Y. Courbois, J. Van Herwegen, M. Blades, How useful are landmarks when learning a route in a virtual environment? Evidence from typical development and Williams syndrome. J Exp Child Psychol 111, 571-586 (2012).
21. H. R. Purser et al., Short-term memory, executive control, and children's route learning. J Exp Child Psychol 113, 273-285 (2012).
22. F. L. Dolins, C. Klimowicz, J. Kelley, C. R. Menzel, Using virtual reality to investigate comparative spatial cognitive abilities in chimpanzees and humans. Am J Primatol, (2014).
23. J. M. Braun et al., Predictors of virtual radial arm maze performance in adolescent Italian children. Neurotoxicology 33, 1203-1211 (2012).
24. V. D. Bohbot et al., Virtual navigation strategies from childhood to senescence: evidence for changes across the life span. Front Aging Neurosci 4, 28 (2012).
25. N. R. Powers et al., Alleles of a polymorphic ETV6 binding site in DCDC2 confer risk of reading and language impairment. Am J Hum Genet 93, 19-28 (2013).
26. N. R. Powers et al., The regulatory element READ1 epistatically influences reading and language, with both deleterious and protective alleles. J Med Genet, (2015).
27. H. Meng et al., DCDC2 is associated with reading disability and modulates neuronal development in the brain. Proc Natl Acad Sci USA 102, 17053-17058 (2005).
28. H. Meng et al., A dyslexia-associated variant in DCDC2 changes gene expression. Behav Genet 41, 58-66 (2011).
29. C. Francks et al., A 77-kilobase region of chromosome 6p22.2 is associated with dyslexia in families from the United Kingdom and from the United States. Am J Hum Genet 75, 1046-1058 (2004).
30. S. Paracchini et al., Association of the KIAA0319 dyslexia susceptibility gene with reading skills in the general population. Am J Psychiatry 165, 1576-1584 (2008).
31. T. S. Scerri et al., DCDC2, KIAA0319 and CMIP are associated with reading-related traits. Biol Psychiatry 70, 237-245 (2011).
32. M. Luciano et al., A haplotype spanning KIAA0319 and TTRAP is associated with normal variation in reading and spelling ability. Biol Psychiatry 62, 811-817 (2007).
33. S. Paracchini et al., The chromosome 6p22 haplotype associated with dyslexia reduces the expression of KIAA0319, a novel gene involved in neuronal migration. Hum Mol Genet 15, 1659-1666 (2006).
34. M. Y. Dennis et al., A common variant associated with dyslexia reduces expression of the KIAA0319 gene. PLoS Genet 5, e1000436 (2009).
35. H. Meng et al., TDT-association analysis of EKN1 and dyslexia in a Colorado twin cohort. Hum Genet 118, 87-90 (2005).
36. V. J. Peschansky et al., The effect of variation in expression of the candidate dyslexia susceptibility gene homolog Kiaa0319 on neuronal migration and dendritic morphology in the rat. Cereb Cortex 20, 884-897 (2010).
37. R. W. Woodcock, K. S. McGrew, N. Mather, Woodcock-Johnson III Tests of Achievement. (Riverside Publishing, Itasca, Ill., 2001).
38. F. A. Schrank, R. W. Woodcock, Woodcock-Johnson III. WJ III Compuscore and Profiles Program (Riverside Publishing, Itasca, Ill., 2001).
39. F. Ramus, C. R. Marshall, S. Rosen, H. K. van der Lely, Phonological deficits in specific language impairment and developmental dyslexia: toward a multidimensional model. Brain 136, 630-645 (2013).
40. D. V. Bishop, M. J. Snowling, Developmental dyslexia and specific language impairment: same or different? Psychol Bull 130, 858-886 (2004).
41. D. L. Lefly, B. F. Pennington, Reliability and validity of the adult reading history questionnaire. J Learn Disabil 33, 286-296 (2000).
42. D. I. Shore, L. Stanford, W. J. MacInnes, R. M. Klein, R. E. Brown, Of mice and men: virtual Hebb-Williams mazes permit comparison of spatial learning across species. Cogn Affect Behav Neurosci 1, 83-89 (2001).
43. J. C. Barrett, B. Fry, J. Maller, M. J. Daly, Haploview: analysis and visualization of LD and haplotype maps. Bioinformatics 21, 263-265 (2005).
44. S. Purcell et al., PLINK: a tool set for whole-genome association and population-based linkage analyses. Am J Hum Genet 81, 559-575 (2007).
45. C. Hulme, M. J. Snowling, Reading disorders and dyslexia. Curr Opin Pediatr 28, 731-735 (2016).
46. A. A. Lauterbach, Y. Park, L. J. Lombardino, The roles of cognitive and language abilities in predicting decoding and reading comprehension: comparisons of dyslexia and specific language impairment. Ann Dyslexia, (2016).
47. H. W. Catts, S. M. Adlof, T. P. Hogan, S. E. Weismer, Are specific language impairment and dyslexia distinct disorders? J Speech Lang Hear Res 48, 1378-1396 (2005).
48. F. Darki, M. Peyrard-Janvid, H. Matsson, J. Kere, T. Klingberg, Three dyslexia susceptibility genes, DYX1C1, DCDC2, and KIAA0319, affect temporo-parietal white matter structure. Biol Psychiatry 72, 671-676 (2012).
49. F. Darki, M. Peyrard-Janvid, H. Matsson, J. Kere, T. Klingberg, DCDC2 polymorphism is associated with left temporoparietal gray and white matter structures during development. J Neurosci 34, 14455-14462 (2014).
50. C. Marino et al., The DCDC2/intron 2 deletion and white matter disorganization: focus on developmental dyslexia. Cortex 57, 227-243 (2014).
51. G. M. Cicchini, C. Marino, S. Mascheretti, D. Perani, M. C. Morrone, Strong Motion Deficits in Dyslexia Associated with DCDC2 Gene Alteration. J Neurosci 35, 8059-8064 (2015).
52. J. D. Eicher et al., Characterization of the DYX2 locus on chromosome 6p22 with reading disability, language impairment, and IQ. Hum Genet, (2014).
53. A. Che, M. J. Girgenti, J. Loturco, The Dyslexia-Associated Gene Dcdc2 Is

Required for Spike-Timing Precision in Mouse Neocortex. Biol Psychiatry, (2013).
54. K. Ludwig et al., Investigation of the DCDC2 intron 2 deletion/compound short tandem repeat polymorphism in a large German dyslexia sample. Psychiatr Genet 18, 310-312 (2008).
55. D. T. Truong et al., Mutation of Dcdc2 in mice leads to impairments in auditory processing and memory ability. Genes Brain Behav 13, 802-811 (2014).
56. R. W. Woodcock, Woodcock reading mastery tests: WRMT-III. (Pearson, ed. 3, 2011).
57. L. S. MacLeod et al., A comparative study of the performance of individuals with fragile X syndrome and Fmr1 knockout mice on Hebb-Williams mazes. Genes Brain Behav 9, 53-64 (2010).

What is claimed is:

1. A method for identifying whether a child has a reading impairment using a virtual rendition of one or more standard Hebb-Williams (HW) mazes comprising:
displaying a virtual HW maze on a display screen associated with a computer running maze simulation software to provide a virtual HW maze navigation experience that emulates a physical HW maze navigation experience, the virtual HW maze having exterior vertical walls and interior vertical walls on a z-axis, the exterior vertical walls and the interior vertical walls extending above a navigational viewing height on the z-axis to enable a three dimensional experience as navigation occurs, the exterior vertical walls defining an external boundary of the virtual HW maze and the interior vertical walls defining corridors, dead ends, or both within the external boundary so that a finish box is not visible from a start box, the start box and the finish box abutting and open to the virtual HW maze;
starting a navigation trial upon exiting the start box and stopping the navigation trial upon entering the finish box or when 120 seconds has elapsed, whichever occurs first;
capturing data points at regular intervals during the navigation trial using the maze simulation software, the captured data points to track time and a path taken as a child virtually navigates through the virtual HW maze, wherein captured time data points are obtained from computer hardware; and
comparing a generated score for the navigation trial to a control score to determine if the generated score is significantly different from the control score, the generated score a function of the captured data points.

2. The method of claim 1 further comprising visualizing the path taken during the navigation trial by using captured data points to map the path taken to a 6×6 grid that divides an interior of the virtual HW maze, excluding the start box and the finish box, into 36 individual cells, the map based on x-axis coordinates and y-axis coordinates associated with the grid and captured time data points.

3. The method of claim 2 further comprising dividing each of the 36 individual cells into 256 units and visualizing the path taken on a unit level.

4. The method of claim 2 further comprising coding each of the 36 individual cells according to a corresponding z-score to provide a dynamic representation of problem solving strategies employed by the child during the navigation trial, the corresponding z-scores derived from time spent and distance traveled within a given cell.

5. The method of claim 4 further comprising examining the dynamic representation and navigation performance to identify a specific reading impairment that the child may have.

6. The method of claim 1 further comprising utilizing at least 4 different virtual HW mazes and 6 separate navigation trials for each of the at least 4 different virtual HW mazes to collect at least 24 sets of captured data points and using the at least 24 sets of captured data points to generate the generated score, wherein if the generated score is greater than one standard deviation away from the control score, identifying the child as reading impaired.

7. The method of claim 6 wherein the at least 4 different virtual HW mazes are based on standard physical HW mazes selected from the group consisting of: Maze 5, Maze 6, Maze 8, Maze 11, and Maze 12.

8. The method of claim 7 further comprising examining a learning rate for the child by evaluating navigation efficiency for each of the 6 separate navigation trials for each of the at least 4 different virtual HW mazes.

9. The method of claim 1 further comprising randomly orienting an initial z-axis view within the start box.

10. The method of claim 9 further comprising blocking reentry into the start box after exiting the start box.

11. The method of claim 1 further comprising displaying, on the display screen, a graphical icon indicative of success if the child navigated through the virtual HW maze from the start box to the finish box in 120 seconds or less, otherwise stop capturing data points and displaying one or more graphical icons to show a way to the finish box.

12. The method of claim 1 wherein the generated score is a performance efficiency score derived from equal weighting of a duration of the navigation trial and an error number that occurred during the navigation trial.

13. The method of claim 1 wherein the virtual HW maze is devoid of landmarks, visual cues, or both.

14. The method of claim 1 further comprising accessing the maze simulation software from a Web-based platform.

15. The method of claim 1 wherein identifying whether the child has a reading impairment includes identifying whether a prereading child, a nonnative language child, or both has a reading impairment.

16. A nonlanguage-based tool to screen children for reading impairments comprising:
a computer having a processor, a memory, and a storage;
a display operatively connected to the computer;
wherein the memory of the computer stores executable code which, when executed, enables the computer to perform a process comprising:
displaying, on the display, a 3D representation of a first standard physical Hebb-Williams (HW) maze selected from the group consisting of: Maze 5, Maze 6, Maze 8, Maze 11, and Maze 12;
capturing data points at regular intervals to track a path taken by a child being screened as the child virtually navigates through the first HW maze being displayed, the child to virtually navigate through the first HW maze being displayed at least 6 consecutive times;
generating an efficiency score for the child using captured data points from the at least 6 consecutive times; and
comparing the generated efficiency score to a control score to determine whether the child has a reading impairment.

17. The tool of claim 16 further comprising after displaying the first HW maze, displaying a second HW maze selected from the group consisting of: Maze 5, Maze 6, Maze 8, Maze 11, and Maze 12; capturing data points at regular intervals to track a path taken by the child as the child virtually navigates through the second HW maze at least 6 consecutive times; and generating the efficiency score using captured data points from the first HW maze navigations and the second HW maze navigations which, when compared to the control score, determines whether the child has a reading impairment.

18. The tool of claim 17 further comprising displaying a two-dimensional representation of each path taken while navigating through the first HW maze and the second HW maze using the captured data points from each path taken to each path taken on a two-dimensional version of each displayed HW maze.

19. A method for detecting reading impairments in young children comprising:
generating an efficiency score relating to the efficiency in which a given child has virtually navigated four different mazes displayed on a display screen to provide a virtual, three-dimensional maze navigation experience of a standard physical Hebb-Williams (HW) maze, each of the four different mazes having been navigated by the given child on six separate occasions, the generated efficiency score derived from data points captured by maze simulation software; and
comparing the generated efficiency score to a control score to determine whether the given child is at risk for a reading impairment.

20. The method of claim 19 further comprising using the captured data points to map a path taken for each time each selected HW maze was navigated and to generate a heat map for each HW maze navigated.
